(19) United States
(12) Patent Application Publication
(10) Pub. No.: US 2003/0077603 A1
(43) Pub. Date: Apr. 24, 2003

(54) Methods and compositions for diagnosis of hepatoma

(76) Inventors: Rajesh Bhikhu Parekh, Wendlebury (GB); Thakorbhai Parshotambhai Patel, Horton-cum-Studley (GB); Robert Reid Townsend, Oxford (GB); Christopher Moyses, Great Milton (GB); Philip J. Johnson, Shatin (HK)

Correspondence Address: PENNIE AND EDMONDS, 1155 AVENUE OF THE AMERICAS, NEW YORK, NY, 100362711

(21) Appl. No.: 10/079,596

(22) Filed: Feb. 19, 2002

Related U.S. Application Data
(63) Continuation-in-part of application No. 09/637,681, filed on Aug. 14, 2000, now abandoned.
(63) Continuation-in-part of application No. PCT/GB00/03224, filed on Aug. 18, 2000.

(30) Foreign Application Priority Data
Aug. 18, 1999 (GB) 9919597.6

(51) Int. Cl. C12Q 1/68; G01N 33/574; G01N 33/561

(52) U.S. Cl. 435/6; 435/7.23; 436/516

(57) ABSTRACT

The present invention provides methods and compositions for screening, diagnosis and prognosis of hepatoma, for monitoring the effectiveness of hepatoma treatment, and for drug development. Hepatoma-Diagnostic Features (HFs), detectable by two-dimensional electrophoresis of serum or plasma are described. The invention further provides Hepatoma-Diagnostic Protein Isoforms (HPIs) detectable in serum or plasma, preparations comprising isolated HPIs, antibodies immunospecific for HPIs, and kits comprising the aforesaid.

### 1. INTRODUCTION

The present invention relates to the identification of proteins and protein isoforms that are associated with hepatocellular carcinoma (hepatoma) and to their use for screening, diagnosis, prognosis, therapy and drug development.

### 2. BACKGROUND OF THE INVENTION

Hepatoma is an increasingly prevalent cancer, affecting several hundred thousand patients worldwide. Methods for its treatment are still relatively poor, and the main hope for more effective therapy lies in earlier and more accurate diagnosis.

Measurement of serum alphafetoprotein (AFP) is widely used in the diagnosis of hepatoma. While AFP is a useful marker when levels are markedly elevated, poor specificity and poor positive predictive value (PPV) for hepatoma at lower levels severely limit its practical application. Johnson et al., 1997, Br. J. Cancer 75: 236-240. Certain lectins show differential binding to AFP from hepatoma serum compared with AFP in benign conditions. Aoyagi et al., 1985, Biochim. Biophys. Acta 830: 217-223; Aoyagi et al., 1993, Br. J. Cancer 67: 486-492. However, these approaches have not found broad clinical acceptance. Johnson et al., supra. More recently, isoelectric focusing has been used to detect isoforms of AFP that appear relatively specific for hepatoma. Johnson et al., supra. These reports illustrate an ongoing need for identification of markers in serum for diagnosis of hepatoma.

### 3. SUMMARY OF THE INVENTION

The present invention provides methods and compositions for screening, diagnosis and prognosis of hepatoma, for monitoring the effectiveness of hepatoma treatment, and for drug development.

A first aspect of the invention provides methods for diagnosis of hepatoma that comprise analyzing a first test sample of plasma or serum by two-dimensional electrophoresis to detect the level of at least one Hepatoma-Diagnostic Feature (HF), e.g., an HF selected from the group of HFs disclosed herein. These methods are also suitable for screening, prognosis, monitoring the results of therapy, and drug development.

A second aspect of the invention provides methods for diagnosis of hepatoma that comprise detecting in a first test sample of plasma or serum the level of at least one Hepatoma-Diagnostic Protein Isoform (HPI), e.g., an HPI selected from the group of HPIs disclosed herein. These methods are also suitable for screening, prognosis, monitoring the results of therapy, and drug development.

A third aspect of the invention provides monoclonal and polyclonal antibodies capable of immunospecific binding to an HPI, e.g., an HPI disclosed herein.

A fourth aspect of the invention provides a preparation comprising an isolated HPI, i.e. an HPI free from proteins or protein isoforms having a significantly different isoelectric point or a significantly different apparent molecular weight from the HPI.

### 4. BRIEF DESCRIPTION OF FIGURES

FIG. 1 is an image obtained from 2-dimensional electrophoresis of normal human serum, which has been annotated to identify 14 landmark features, designated PL1 to PL12 and PL15 to PL16.

### 5. DETAILED DESCRIPTION OF THE INVENTION

The invention described in detail below encompasses methods and compositions for screening, diagnosis and prognosis of hepatoma in a mammalian subject, methods for monitoring the results of hepatoma therapy, and methods for drug development. Preferably, the subject is human, more preferably a human adult.

For clarity of disclosure, and not by way of limitation, the invention will be described with respect to the analysis of serum samples. However, as one skilled in the art will appreciate, the assays and techniques described herein can be applied to other types of patient samples, including a body fluid (e.g. plasma, urine, bile, ascitic fluid), a tissue sample suspected of containing material derived from a hepatoma (e.g. a biopsy such as a liver biopsy or a biopsy of a suspicious mass) or homogenate thereof.

### 5.1. Hepatoma-Diagnostic Features (HFs)

In one aspect of the invention, two-dimensional electrophoresis is used to analyze serum from a subject in order to measure the abundance of one or more Hepatoma-Diagnostic Features (HFs) for screening or diagnosis of hepatoma, to determine the prognosis of a hepatoma patient, to monitor the effectiveness of hepatoma therapy, or for drug development. As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in U.S. Pat. No. 6,064,754 and WO 98/23950 which are incorporated herein by reference in their entirety. Briefly, the Preferred Technology provides efficient, computer-assisted methods and apparatus for identifying, selecting and characterizing biomolecules in a biological sample. A two-dimensional array is generated by separating biomolecules in a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

In certain instances, it may prove desirable to ‘process’ a biological sample before protein separation is performed with a view either to selectively enriching certain desirable proteins from within the sample or to selectively depleting the sample of certain undesirable proteins. For example, if glycosylated proteins only are of interest, such glycoproteins may be selectively isolated from a sample using lectin-affinity chromatography or lectin affinity precipitation.

Such enrichment can both enhance and simplify the subsequent protein separation and analysis. Any proteins or group of proteins carrying a structural determinant for which an antibody or other specific purification reagent is available may be so extracted, e.g. tyrosine phospho-proteins using an anti-phosphotyrosine antibody. Conversely, a sample may be depleted of specific proteins again using protein-specific affinity methods. For example, albumin may be removed from body fluids using an anti-albumin antibody, immunoglobulins may be removed using protein A or protein G (preferably immobilised). It is clear that a sample may be selectively depleted (or enriched) for more than one protein by using protein-specific reagents serially or in combination. Such enrichment/depletion can often have a beneficial effect during analysis by concentrating proteins of interest/removing proteins that interfere with or may, for example by their predominance, limit the analysis of proteins of interest.

As used herein, the term “Hepatoma-Diagnostic Feature” (HF) refers to a feature (e.g. a spot in a 2D gel), detectable by 2D electrophoresis of a biological sample, that is differentially present in a first test sample compared to second, relevant sample, e.g., in serum from a subject with hepatoma compared with serum from a subject without hepatoma. As used herein, a feature (or a protein isoform) is “differentially present” in a first sample with respect to a second sample when a method for detecting the feature or isoform (e.g. 2D electrophoresis or an immunoassay) reveals that the feature (or protein isoform) is present at a different relative abundance in the first sample as compared with the second sample. If the measured feature in the first sample is at a higher abundance than in the second sample, the feature or isoform is “increased” in the first sample with respect to the second; conversely, if the measured feature in the first sample is at a lower abundance than in the second sample, the feature or isoforn is “decreased” in the first sample with respect to the second.

Preferably, the relative abundance of a feature in two samples is determined in two steps. First, the signal obtained upon detecting the feature in a sample is normalized by reference to a suitable background parameter, e.g., to the total protein in the sample being analyzed (e.g., total protein loaded onto a gel), to an invariant feature, i.e., a feature whose abundance is known to be similar in the samples being compared, e.g., one or more Expression Reference Features (ERFs), such as the ERFs disclosed below, or to the total signal detected from all proteins in the sample.

Secondly, the normalized signal for the feature in one sample or sample set is compared with the normalized signal for the same feature in another sample or sample set in order to identify features that are “differentially present” in the first sample (or sample set) with respect to the second.

By way of example and not of limitation, using the Preferred Technology, a number of samples from subjects having hepatoma and samples from subjects free from hepatoma are separated by two-dimensional electrophoresis, and the fluorescent digital images of the resulting gels are matched to a chosen representative primary master gel image. This process allows any gel feature, characterised by its pl and MW, to be identified and examined on any gel of the study. In particular, the amount of protein present in a given feature can be measured in each gel; this feature abundance can be averaged amongst gels from similar samples (e.g. gels from samples from subjects having hepatoma). Finally, statistical analyses can be conducted on the thus created sample sets, in order to compare 2 or more sample sets to each other.

Two groups of HFs have been identified through the methods and apparatus of the Preferred Technology. The first group consists of HFs that are decreased in the serum of subjects with hepatoma as compared with the serum of subjects without hepatoma (e.g. subjects with cirrhosis). These HFs can be described by apparent molecular weight (MW) and isoelectric point (pI) as follows:

TABLE I
HFs Decreased In Hepatoma Serum

| | | Fold | | MW | |
|---|---|---|---|---|---|
| Name | MCI | decrease | pI | (Da) | p value |
| HF-1 | 7537 | −11.7 | 8.12 | 44,093 | 0.0003 |
| HF-2 | 7341 | −4.9 | 6.02 | 62,370 | 0.0239 |
| HF-3 | 7232 | −4.6 | 7.60 | 103,154 | 0.0094 |
| HF-4 | 7534 | −3.1 | 7.41 | 44,764 | 0.0278 |
| HF-5 | 7763 | −2.7 | 6.93 | 67,555 | 0.0362 |
| HF-6 | 7945 | −2.7 | 5.73 | 57,295 | 0.0377 |
| HF-7 | 7760 | −2.5 | 6.37 | 69,194 | 0.0109 |
| HF-8 | 7179 | −2.5 | 5.82 | 166,395 | 0.0138 |
| HF-9 | 8031 | −2.1 | 6.15 | 24,467 | 0.0258 |
| HF-10 | 7831 | −2.0 | 7.39 | 37,863 | 0.0025 |
| HF-11 | 7910 | −2.0 | 7.79 | 93,302 | 0.1078 |
| HF-12 | 7562 | −2.0 | 5.37 | 40,672 | 0.0166 |
| HF-13 | 7762 | −2.0 | 6.41 | 67,826 | 0.1015 |
| HF-14 | 7641 | −2.0 | 6.80 | 28,169 | 0.0364 |
| HF-57 | 679904 | −4.6 | 5.90 | 67,315 | 0.0247 |
| HF-58 | 679924 | −2.3 | 6.12 | 69,018 | 0.0154 |
| HF-59 | 679916 | −2.1 | 5.80 | 67,315 | 0.001057 |
| HF-62 | 680563 | −1.46 | 7.08 | 23722 | |
| HF-63 | 679854 | −1.73 | 6.13 | 80395 | 0.00112 |
| HF-64 | 679928 | −1.59 | 5.75 | 67737 | |
| HF-65 | 679935 | −2.18 | 5.75 | 65450 | |
| HF-66 | 679958 | −1.21 | 6.06 | 61681 | 0.2706 |
| HF-67 | 679950 | −1.11 | 6.14 | 62261 | 0.4850 |
| HF-68 | 680016 | −1.16 | 6.65 | 55123 | 0.1796 |
| HF-69 | 680040 | −1.25 | 5.32 | 51786 | 0.3336 |
| HF-70 | 680085 | −1.39 | 5.75 | 47898 | 0.03086 |
| HF-71 | 680093 | −1.39 | 5.87 | 47156 | 0.5101 |
| HF-72 | 680135 | −1.29 | 5.31 | 42806 | 0.02175 |
| HF-73 | 680175 | −1.34 | 4.96 | 37545 | 0.02337 |
| HF-74 | 680338 | −1.22 | 5.44 | 25022 | 0.5049 |
| HF-75 | 680345 | −1.74 | 7.5 | 24178 | 0.0001741 |
| HF-76 | 680524 | −1.46 | 5.18 | 51865 | 0.04133 |
| HF-77 | 680700 | −1.31 | 7.24 | 114862 | 0.1913 |
| HF-78 | 680563 | −1.46 | 7.08 | 23722 | |
| HF-79 | 680087 | −1.39 | 6.14 | 47898 | |
| HF-80 | 679971 | −1.69 | 5.33 | 61297 | 0.09826 |
| HF-81 | 680229 | −1.87 | 5.2 | 32931 | 0.4074 |
| HF-82 | 680624 | −2.01 | 5.78 | 61255 | 0.2437 |
| HF-83 | 680342 | −2.61 | 5.22 | 24481 | 0.02259 |
| HF-84 | 680371 | −1.54 | 5.22 | 22851 | 0.00002744 |
| HF-85 | 680372 | −1.64 | 5.09 | 23289 | 0.00006081 |
| HF-86 | 682292 | −1.76 | 5.12 | 24646 | |
| HF-87 | 680318 | −5.51 | 5.21 | 25977 | 0.07947 |
| HF-88 | 681606 | −33.73 | 9.83 | 10844 | |
| HF-89 | 679858 | −1.42 | 6.06 | 79315 | 0.0007902 |
| HF-90 | 679888 | −1.55 | 4.76 | 73465 | 0.01347 |
| HF-91 | 680386 | −1.28 | 5.6 | 21621 | 0.0006296 |
| HF-92 | 680446 | −1.18 | 5.86 | 13845 | 0.5442 |
| HF-93 | 680181 | −1.13 | 5.14 | 37311 | 0.5343 |
| HF-94 | 680291 | −1.98 | 5.53 | 27307 | 0.03241 |
| HF-95 | 680378 | −2.17 | 5.76 | 23144 | |
| HF-96 | 680406 | −1.19 | 5.97 | 19045 | 0.3209 |
| HF-97 | 680409 | −1.67 | 5.79 | 18798 | 0.0008859 |
| HF-98 | 680429 | −1.14 | 4.47 | 14728 | 0.5350 |
| HF-99 | 682264 | −4.81 | 7.64 | 46831 | |
| HF-100 | 679814 | −1.48 | 6 | 89983 | 0.002941 |
| HF-101 | 680036 | −1.78 | 5.78 | 52437 | 0.07205 |
| HF-102 | 680208 | −1.43 | 5.87 | 34835 | 0.1301 |
| HF-103 | 680628 | −3.07 | 5.58 | 53701 | 0.004082 |

The second groups consists of HFs that are increased in the serum of subjects with hepatoma as compared with the serum of subjects without hepatoma (e.g. subjects with cirrhosis). These HFs can be described by apparent molecular weight (MW) and isoelectric point (pI) as follows:

TABLE II
HFs Increased In Hepatoma Serum

| | | Fold | | MW | |
|---|---|---|---|---|---|
| Name | MCI | increase | pI | (Da) | p-value |
| HF-15 | 7308 | 3.9 | 5.55 | 67,555 | 0.0013 |
| HF-16 | 7463 | 3.8 | 7.84 | 50,625 | 0.0055 |
| HF-17 | 7629 | 3.1 | 7.27 | 31,244 | 0.0000 |
| HF-18 | 7366 | 3.0 | 4.60 | 59,928 | 0.0000 |
| HF-19 | 7759 | 3.0 | 5.40 | 69,194 | 0.0021 |
| HF-20 | 7581 | 3.0 | 4.94 | 38,465 | 0.0000 |
| HF-21 | 7320 | 2.9 | 7.63 | 66,485 | 0.0009 |
| HF-22 | 7690 | 2.9 | 5.72 | 16,707 | 0.0136 |
| HF-23 | 7241 | 2.8 | 5.27 | 96,988 | 0.0000 |
| HF-24 | 7242 | 2.7 | 5.31 | 96,988 | 0.0000 |
| HF-25 | 7240 | 2.6 | 5.14 | 97,732 | 0.0000 |
| HF-26 | 7734 | 2.6 | 6.04 | 108,311 | 0.0011 |
| HF-27 | 7469 | 2.6 | 8.12 | 50,134 | 0.0344 |
| HF-28 | 7431 | 2.6 | 5.34 | 52,432 | 0.0009 |
| HF-29 | 7553 | 2.5 | 4.84 | 41,288 | 0.0000 |
| HF-30 | 7457 | 2.5 | 7.61 | 50,822 | 0.0255 |
| HF-31 | 7330 | 2.4 | 7.44 | 63,882 | 0.0003 |
| HF-32 | 7460 | 2.4 | 7.25 | 50,625 | 0.0085 |
| HF-33 | 7237 | 2.4 | 5.10 | 97,732 | 0.0000 |
| HF-34 | 7243 | 2.4 | 5.21 | 96,988 | 0.0000 |
| HF-35 | 7368 | 2.3 | 4.57 | 60168 | 0.0168 |
| HF-36 | 7730 | 2.3 | 5.10 | 136,409 | 0.0007 |
| HF-37 | 7288 | 2.3 | 6.79 | 71,726 | 0.0035 |
| HF-38 | 7568 | 2.3 | 5.34 | 39,869 | 0.0034 |
| HF-39 | 7238 | 2.3 | 5.04 | 97,732 | 0.0000 |
| HF-40 | 7691 | 2.3 | 5.40 | 16,551 | 0.0049 |
| HF-41 | 7317 | 2.2 | 6.06 | 66,220 | 0.0974 |
| HF-42 | 7465 | 2.2 | 7.44 | 50,329 | 0.0097 |
| HF-43 | 7380 | 2.2 | 6.01 | 58,044 | 0.0144 |
| HF-44 | 7239 | 2.2 | 5.07 | 97,732 | 0.0000 |
| HF-45 | 7692 | 2.2 | 6.10 | 16,447 | 0.0412 |
| HF-46 | 7379 | 2.2 | 4.66 | 57,582 | 0.0040 |
| HF-47 | 7180 | 2.2 | 5.36 | 164,341 | 0.0059 |
| HF-48 | 7633 | 2.2 | 7.26 | 30,638 | 0.0016 |
| HF-49 | 7392 | 2.1 | 5.10 | 56,444 | 0.0014 |
| HF-50 | 7572 | 2.1 | 5.71 | 39,239 | 0.0227 |
| HF-51 | 7190 | 2.0 | 5.26 | 161,308 | 0.0001 |
| HF-52 | 7186 | 2.0 | 5.32 | 163,324 | 0.0008 |
| HF-53 | 7556 | 2.0 | 5.15 | 40,753 | 0.0020 |
| HF-54 | 7540 | 2.0 | 5.29 | 43,541 | 0.0004 |
| HF-55 | 7185 | 2.0 | 5.22 | 163,324 | 0.0000 |
| HF-56 | 7822 | 2.0 | 5.12 | 42,416 | 0.0030 |
| HF-60 | 680238 | 2.2 | 7.28 | 31,919 | 0.003 |
| HF-61 | 680247 | 2.0 | 7.5 | 30,841 | 0.012 |
| HF-104 | 679918 | 1.28 | 4.58 | 65860 | 0.06848 |
| HF-105 | 679868 | 1.38 | 8.78 | 76852 | 0.05490 |
| HF-106 | 679871 | 1.28 | 8.65 | 75820 | 0.1096 |
| HF-107 | 680003 | 2.02 | 6.63 | 57407 | 0.07747 |
| HF-108 | 680028 | 1.26 | 6.54 | 52931 | 0.07665 |
| HF-109 | 680137 | 2.42 | 4.97 | 42143 | 0.001191 |
| HF-110 | 680142 | 2.42 | 4.87 | 41880 | 0.0003546 |
| HF-111 | 680177 | 1.66 | 5.33 | 37428 | 0.05373 |
| HF-112 | 680218 | 1.77 | 5.21 | 33764 | 0.001107 |
| HF-113 | 680238 | 2.17 | 7.28 | 31919 | 0.0002972 |
| HF-114 | 680280 | 1.13 | 6.85 | 27650 | 0.3030 |
| HF-115 | 680418 | 1.99 | 5.71 | 16344 | 0.02678 |
| HF-116 | 680425 | 1.95 | 7.03 | 14969 | 0.01694 |
| HF-117 | 680653 | 1.83 | 8.25 | 28212 | |
| HF-118 | 679939 | 2.07 | 7.74 | 64436 | 0.01761 |
| HF-119 | 679972 | 1.9 | 5.28 | 61105 | 0.01859 |
| HF-120 | 679757 | 2.29 | 6.72 | 108464 | 0.0001996 |
| HF-121 | 679762 | 2.1 | 6.6 | 107212 | 0.0004064 |
| HF-122 | 679766 | 1.66 | 6.48 | 104146 | 0.0003548 |
| HF-123 | 679876 | 1.86 | 7.75 | 75479 | 0.01350 |
| HF-124 | 680075 | 1.84 | 4.13 | 45280 | 0.0002768 |
| HF-125 | 680124 | 1.13 | 6.75 | 43752 | |
| HF-126 | 680244 | 1.73 | 7.67 | 31035 | 0.1775 |
| HF-127 | 680250 | 1.45 | 7.28 | 30745 | 0.07981 |
| HF-128 | 680427 | 1.29 | 8.35 | 14872 | |
| HF-129 | 680599 | 1.63 | 6.82 | 104310 | 0.03921 |
| HF-130 | 680783 | 16.39 | 5.43 | 65816 | |
| HF-131 | 680815 | 13.63 | 6.96 | 26468 | |
| HF-132 | 681676 | 8.81 | 7.7 | 34209 | |
| HF-133 | 681924 | 8.92 | 7.02 | 11712 | |
| HF-134 | 680362 | 1.25 | 6.59 | 24178 | 0.7421 |
| HF-135 | 680171 | 1.22 | 5.13 | 37898 | 0.2304 |
| HF-136 | 680275 | 1.22 | 7.89 | 28085 | 0.1920 |
| HF-137 | 680424 | 1.38 | 8.34 | 15464 | 0.1933 |
| HF-138 | 680476 | 1.25 | 7.33 | 10777 | 0.4495 |
| HF-139 | 681923 | 10.69 | 6.81 | 11858 | |
| HF-140 | 680147 | 1.4 | 4.88 | 41232 | |
| HF-141 | 680101 | 1.2 | 6.65 | 46571 | 0.3139 |

For any given HF, the ratio obtained upon comparing the normalized signal observed upon analyzing serum from subjects with hepatoma relative to the normalized signal obtained upon analyzing serum from subjects without hepatoma will depend upon the particular analytical protocol and detection technique that is used. Accordingly, the present invention contemplates that each laboratory will establish a reference range for each HF in hepatoma-free subjects according to the analytical protocol and detection technique in use, as is conventional in the diagnostic art. Preferably, at least one positive control serum sample from a subject known to have hepatoma or at least one negative control serum sample from a subject known to be free of hepatoma (and more preferably at least one positive and at least one negative control sample) is included in each batch of test samples analyzed. In one embodiment, the level of expression of a feature is determined relative to a background value, which is defined as the level of signal obtained from a proximal region of the image that (a) is equivalent in area to the particular feature in question; and (b) contains no substantial discermable protein feature.

As the skilled artisan will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight and the isoelectric point of a feature or protein isoform as measured in exact accordance with the experimental protocol set forth in Section 6 below (“the Reference Protocol”). When the Reference Protocol is followed and when samples are run in duplicate or a higher number of replicates, variation in the measured mean pI of an HF or HPI is typically less than ±1% and variation in the measured mean MW of an HF or HPI is typically less than ±5%. Where the skilled artisan wishes to deviate from the Reference Protocol, calibration experiments should be performed to compare the MW and pI for each HF or protein isoform as detected (a) by the Reference Protocol and (b) by the deviant protocol.

HFs can be used for detection, prognosis, diagnosis, or monitoring of hepatoma or for drug development. In one embodiment of the invention, serum from a subject is analyzed by 2D electrophoresis for quantitative detection of one or more HFs selected from the group consisting of HF-1 to HF-14, HF-57 to HF-59, and HF-62 to HF-103 wherein a decreased abundance of an HF in serum from the subject relative to serum from a subject (or subjects) without hepatoma (e.g., a control sample or a previously determined reference range) indicates the presence of hepatoma; preferably, the one or more HFs are selected from the group consisting of HF-1, HF-59, HF-75, HF-84, HF-85, HF-89, HF-91, HF-97. In another embodiment of the invention, serum from a subject is analyzed by 2D electrophoresis for quantitative detection of one or more HFs selected from the group consisting of HF-15 to HF-56 and HF-60 to HF-61, and HF-104 to HF-141 wherein an increased abundance of an HF in serum from the subject relative to serum from a subject (or subjects) without hepatoma (e.g., a control sample or a previously determined reference range) indicates the presence of hepatoma; preferably, the one or more HFs are selected from the group consisting of HF-17, HF-18, HF-20, HF-21, HF-23, HF-24, HF-25, HF-28, HF-29, HF-31, HF-33, HF-34, HF-36, HF-39, HF-44, HF-51, HF-52, HF-54 and HF-55; HF-110, HF-113, HF-120, HF-121, HF-122, still more preferably, the one or more HFs are selected from the group consisting of HF-17, HF-18, HF-20, HF-23, HF-24, HF-25, HF-29, HF-33, HF-34, HF-39, HF-44 and HF-55.

In a preferred embodiment a serum sample from a patient is analysed for the quantitative detection of a plurality of HFs.

### 5.2. Hepatoma-Diagnostic Protein Isoforms (HPIs)

In another aspect of the invention, serum from a subject is analyzed for quantitative detection of one or more “Hepatoma-Diagnostic Protein Isoforms” (HPIs) for screening or diagnosis of hepatoma, to determine the prognosis of a hepatoma patient, to monitor the effectiveness of hepatoma therapy, or for drug development. As used herein, the term “Hepatoma-Diagnostic Protein Isoform” refers to a protein isoform that is differentially present in serum subjects with hepatoma compared with serum from subjects without hepatoma. As is well known in the art, the protein product of a single gene may be expressed as variants (isoforms) (a) that differ as a result of differential post-translational modification (e.g. glycosylation, phosphorylation or acylation), so that proteins of identical amino acid sequence can differ in their pI, MW or both, and/or (b) that differ in their amino acid composition (e.g. as a result of alternative mRNA splicing or limited proteolysis). It follows that differential presence of a protein isoform does not require differential expression of the gene encoding the protein in question.

Two groups of HPIs have been identified by partial amino acid sequencing of HFs, using the methods and apparatus of the Preferred Technology. The first group consists of HPIs that are decreased in the serum of subjects with hepatoma as compared with the serum of subjects without hepatoma. The MW, pI and partial amino acid sequence of these HPIs are presented in Table III, as follows:

TABLE III
HPI Decreased In Hepatoma Serum

| | | Known | | | |
|---|---|---|---|---|---|
| | | homologous | Partial amino acid | | MW |
| HF # | HPI | protein | sequence | pI | (kD) |
| 1 | HPI-1 | Immuno- | REPQVYTLPPSR | 8.12 | 44,093 |
| | | globulin | (SEQ ID NO.1) | | |
| | | G- H₁, | | | |
| | | H₂, H₃ | | | |
| 57 | HPI-23 | Hemopexin | DYFMPCPGR | 5.90 | 67,315 |
| | | | (SEQ ID NO:2) | | |
| 58 | HPI-24 | C4 Binding | FSAICQGDGTWSPR | 6.12 | 69,018 |
| | | Protein | (SEQ ID NO:3) | | |
| | | Alpha Chain | | | |
| 59 | HPI-25 | Hemopexin | WLQGSQELPR | 5.80 | 67,315 |
| | | | (SEQ ID NO:4 | | |

The second group comprises HPIs that are increased in the serum of subjects with hepatoma as compared with the serum of subjects without hepatoma. The MWs, pIs and partial amino acid sequences of these HPIs are presented in Table IV as follows. For some HPIs, the partial sequence information derived from tandem mass spectrometry was not found to be described in any known public database. These are listed as ‘NOVEL’ in Table IV, and the partial amino acid sequence information for these HPIs is given in Table VI.

TABLE IV
HPIs Increased In Hepatoma Serum

| | | Known homologous | | | | MW |
|---|---|---|---|---|---|---|
| HF# | HPI | protein | Partial amino acid sequence | | pI | (kD) |
| 17 | HPI-2 | complement factor 4 | RGLQDEDGYR | (SEQ ID NO:5) | 7.27 | 31,244 |
| 18 | HPI-3 | α₁-anti-chymotrypsin | KITLLSALVETR | (SEQ ID NO:6) | 4.60 | 59,928 |
| 20 | HPI-4 | complement factor 3 | KGYTQQLAFR | (SEQ ID NO:7) | 4.94 | 38,465 |
| 21 | HPI-5 | complement factor 3 | RIPIEDGSGEVVLSR | (SEQ ID NO:8) | 7.63 | 66,485 |
| 23 | HPI-6 | complement factor 4 | RTYNVLDMK | (SEQ ID NO:9) | 5.27 | 96,988 |
| 25 | HPI-8 | complement factor 4 | KAEMADQASAWLTR | (SEQ ID NO:10) | 5.14 | 97,732 |
| 28 | HPI-9 | vitamin D-binding | KHLSLLTTLSNR | (SEQ ID NO:11) | 5.34 | 52,432 |
| | | protein | | | | |
| 29 | HPI-10 | haptoglobin-1 | RVGYVSGWGR | (SEQ ID NO:12) | 4.84 | 41,288 |
| 29 | HPI-11 | haptoglobin-2 | KYVMLPVADQDQCIR | (SEQ ID NO:13) | 4.84 | 41,288 |
| 31 | HPI-12 | complement factor 3 | KTIYTPGSTVLYR | (SEQ ID NO:14) | 7.44 | 63,882 |
| 31 | HPI-13 | NOVEL | See TABLE VI | | 7.44 | 63,882 |
| 33 | HPI-14 | complement factor 4 | RQGSFQGGFR | (SEQ ID NO:15) | 5.10 | 97,732 |
| 34 | HPI-15 | complement factor 4 | RQGSFQGGFR | (SEQ ID NO:15) | 5.21 | 96,988 |
| 36 | HPI-16 | inter-α-trypsin | RFAHTVVTSR | (SEQ ID NO:16) | 5.10 | 136,409 |
| | | inhibitor family | | | | |
| | | heavy chain related | | | | |
| | | protein | | | | |
| 39 | HPI-17 | complement factor 4 | RQGSFQGGFR | (SEQ ID NO:15) | 5.04 | 97,732 |
| 44 | HPI-18 | complement factor 4 | RTYNVLDMK | (SEQ ID NO:9) | 5.07 | 97,732 |
| 51 | HPI-19 | Ceruloplasmin | KGAYPLSIEPIGVR | (SEQ ID NO:17) | 5.26 | 161,308 |
| 52 | HPI-20 | Ceruloplasmin | KALYLQYTDETFR | (SEQ ID NO:18) | 5.32 | 163,324 |
| 54 | HPI-21 | NOVEL | See TABLE VI | | 5.29 | 43,541 |
| 55 | HPI-22 | Ceruloplasmin | RQSEDSTFYLGER | (SEQ ID NO:19) | 5.22 | 163,324 |
| 60 | HPI-26 | Complement | VHYTVCIWR | (SEQ ID NO:20) | 7.28 | 31,919 |
| | | component C4A | | | | |
| 61 | HPI-27 | Complement | CSVFYGAPSK | (SEQ ID NO:21) | 7.5 | 30,841 |
| | | component C4A | | | | |

In one embodiment of the invention, serum from a subject is analyzed for quantitative detection of one or more HPIs selected from the group consisting of HPI-1 and HPI-23 to HPI-25, wherein a decreased level of the one or more HPIs in serum from the subject relative to serum from a subject or subjects without hepatoma (e.g. a control sample or a previously determined reference range) indicates the presence of hepatoma. In another embodiment of the invention, serum from a subject is analyzed by 2D electrophoresis for quantitative detection of one or more HPIs selected from the group consisting of HPI-2 to HPI-22, HPI-26 and HPI-27, wherein an increased level of the one or more HPIs in serum from the subject relative to serum from a subject or subjects without hepatoma (e.g. a control sample or a previously determined reference range) indicates the presence of hepatoma.

As shown above, the HPIs described herein include previously unknown proteins as well as isoforms of known proteins where the isoforms were not previously known to be associated with hepatoma. For each HPI, the present invention additionally provides a preparation comprising the isolated HPI or fragments thereof, and further provides antibodies that bind to said HPI, to said fragments, or to both said HPI and said fragments. As used herein, an “isolated” HPI is an HPI free of proteins or protein isoforms having a significantly different pI or MW from those of the HPI, as determined by 2D electrophoresis. As used herein, a “significantly different” pI or MW is one that causes the contaminating protein isoform to be resolved from the HPI on 2D electrophoresis, performed according to the Reference Protocol.

In one embodiment, an isolated protein is provided, said protein comprising a peptide with the amino acid sequence identified in Table III or IV for an HPI, said protein having a pI and MW within 10% (preferably within 5%, more preferably within 1%) of the values identified in Tables III and IV for that HPI.

The HPIs of the invention can be assayed by any method known to those skilled in the art. In one embodiment, the HPIs are separated on a 2-D gel by virtue of their MWs and pIs and visualized by staining the gel.

Alternatively, HPIs can be detected in assays, such as immunoassays, for detection, prognosis, diagnosis or monitoring of hepatoma or for drug development. In one embodiment, an immunoassay is performed by contacting a sample, derived from a subject to be tested, with an anti-HPI antibody under conditions such that immunospecific binding can occur, and detecting or measuring the amount of any immunospecific binding by the antibody. Preferably, the anti-HPI antibody preferentially binds to the HPI rather than to other isoforms of the same protein. In a preferred embodiment, the anti-HPI antibody binds to the HPI with at least 2-fold greater affinity, more preferably at least 5-fold greater affinity, still more preferably at least 10-fold greater affinity, than to other isoforms of the same protein.

In one embodiment, binding of antibody in tissue sections can be used to detect aberrant HPI localization or aberrant (e.g., high, low, absent) levels of an HPI. In a specific embodiment, antibody to an HPI can be used to assay a patient tissue (e.g. a liver biopsy) or serum sample for the presence of the HPI where an aberrant level of HPI is indicative of hepatoma. As used herein, an “aberrant level” means an increased or decreased level relative to that present, or a standard level representing that present, in an analogous sample from a portion of the body or from a subject not having hepatoma.

The immunoassays which can be used include without limitation competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

If desired, an HPI can be detected by means of a two-step sandwich assay. Where an HPI represents a particular glycoform of a protein, the first step can employ an anti-HPI antibody (which can optionally be immobilized on a solid phase) to capture the HPI; in the second step, a directly or indirectly labelled lectin can be used to detect the captured HPI. Any lectin can be used for this purpose that preferentially binds to the HPI rather than (a) to other glycoforms that have the same core protein as the HPI or (b) to other isoforms that share the antigenic determinant recognized by the antibody. In a preferred embodiment, the chosen lectin binds to the HPI with at least 2-fold greater affinity, more preferably at least 5-fold greater affinity, still more preferably at least 1 0-fold greater affinity, than to said other glycoforms that have the same core protein as the HPI or to said other isoforms that share the same antigenic determinant recognized by the antibody. A lectin that is suitable for detecting a given HPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety).

If desired, a gene encoding an HPI, a related gene and related nucleic acid sequences and subsequences, including complementary sequences, can also be used in hybridization assays. A nucleotide encoding an HPI, or subsequences thereof comprising about at least 8 nucleotides (or the complement of the foregoing) can be used as hybridization probes. Hybridization assays can be used for detection, prognosis, diagnosis, or monitoring of conditions, disorders, or disease states, associated with aberrant changes in HPI gene expression, in particular hepatoma or recrudescence of hepatoma following surgical or other therapy. In a particular embodiment, such a hybridization assay is carried out by a method comprising contacting a patient sample containing nucleic acid with a nucleic acid probe capable of hybridizing to a DNA or RNA encoding an HPI, under conditions such that hybridization can occur, and detecting or measuring any resulting hybridization.

The invention also provides diagnostic kits, comprising in one or more containers an anti-HPI antibody. In addition, such a kit may optionally comprise one or more of the following: (1) instructions for using the anti-HPI antibody for diagnosis, prognosis, therapeutic monitoring, drug development or any combination of these applications; (2) a regulatory notice, i.e. a notice in the form prescribed or approved by a governmental agency regulating the manufacture, use or sale of pharmaceuticals or biological products, which notice reflects approval by the agency of manufacture, use or sale for human testing or administration (3) a labeled binding partner to the antibody; and (4) a solid phase (such as a reagent strip) upon which the anti-HPI antibody is immobilized. If no labeled binding partner to the antibody is provided, the anti-HPI antibody itself can be labeled with a detectable marker, e.g., a chemiluminescent, enzymatic, fluorescent, or radioactive moiety.

The invention also provides a kit comprising in one or more containers a nucleic acid probe capable of hybridizing to RNA encoding a distinct HPI. In a specific embodiment, a kit can comprise in one or more containers a pair of primers (e.g., each in the size range of 6-30 nucleotides, more preferably 10-20 nucleotides) that are capable of priming amplification such as by polymerase chain reaction (see e.g., Innis et al., 1990, PCR Protocols, Academic Press, Inc., San Diego, Calif.), ligase chain reaction (see EP 320,308) use of Qβ replicase, cyclic probe reaction, or other methods known in the art under appropriate reaction conditions of at least a portion of a nucleic acid encoding an HPI.

Kits are also provided which allow for the detection of a plurality of HPIs or a plurality of nucleic acids each encoding an HPI. A kit can optionally further comprise a predetermined amount of an isolated HPI protein or a nucleic acid encoding an HPI, e.g., for use as a standard or control.

### 5.3. Statistical Techniques for Identifying HF and HPI Clusters

Uni-variate differential analysis tools, such as fold changes, Wilcoxon rank sum test and t-test, are useful in identifying individual HFs or HPIs that are diagnostically associated with hepatoma or in identifying individual HPIs that regulate the disease process. However, those skilled in the art will appreciate that the disease process is associated with a suitable combination of HFs or HPIs (and to be regulated by a suitable combination of HPIs), rather than individual HFs and HPIs in isolation. The strategies for discovering such suitable combinations of HFs and HPIs differ from those for discovering individual HFs and HPIs. In such cases, each individual HF and HPI can be regarded as one variable and the disease can be regarded as a joint, multi-variate effect caused by interaction of these variables.

The following steps can be used to identify markers from data produced by the Preferred Technology.

The first step is to identify a collection of HFs or HPIs that individually show significant association with hepatoma. The association between the identified individual HFs or individual HPIs and hepatoma need not be as highly significant when a collection of HFs and HPIs is used as a diagnostic as is desirable when an individual HF or HPI is used as a diagnostic. Any of the tests discussed above (fold changes, Wilcoxon rank sum test, etc.) can be used at this stage. Once a suitable collection of HFs or HPIs has been identified, a sophisticated multi-variate analysis capable of identifying clusters can then be used to estimate the significant multivariate associations with Hepatoma.

Linear Discriminant Analysis (LDA) is one such procedure, which can be used to detect significant association between a cluster of variables (i.e., HFs or HPIs) and Hepatoma. In performing LDA, a set of weights is associated with each variable (i.e., HF or HPI) so that the linear combination of weights and the measured values of the variables can identify the disease state by discriminating between subjects having hepatoma and subjects free from hepatoma. Enhancements to the LDA allow stepwise inclusion (or removal) of variables to optimize the discriminant power of the model. The result of the LDA is therefore a cluster of HFs or HPIs that can be used for diagnosis, treatment or development of pharmaceutical products. Other enhanced variations of LDA, such as Flexible Discriminant Analysis permit the use of non-linear combinations of variables to discriminate a disease state from a state in which there is no disease. The results of the discriminant analysis can be verified by post-hoc tests and also by repeating the analysis using alternative techniques such as classification trees.

A further category of HFs or HPIs can be identified by qualitative measures by comparing the percentage feature presence of an HF or HPI of one group of samples (e.g., samples from diseased subjects) with the percentage feature presence of an HF or HPI in another group of samples (e.g., samples from control subjects). The “percentage feature presence” of an HF or HPI is the percentage of samples in a group of samples in which the HF or HPI is detectable by the detection method of choice. For example, if an HF is detectable in 95 percent of samples from diseased subjects, the percentage feature presence of that HF in that sample group is 95 percent. If only 5 percent of samples from non-diseased subjects have detectable levels of the same HF, detection of that HF in the sample of a subject would suggest that it is likely that the subject has hepatoma

### 5.4. Use in Clinical Studies

The diagnostic methods and compositions of the present invention can assist in conducting or monitoring a clinical study, e.g., for testing drugs for therapy of hepatoma. In one embodiment, candidate molecules are tested for their ability to restore HF or HPI levels in a patient suffering from hepatoma towards levels found in subjects not suffering from hepatoma or, in a treated patient (e.g. after surgery) to maintain HF or HPI levels at or near non-hepatoma values. The levels of one or more HFs or HPIs can be assayed.

In another embodiment, the methods and compositions of the present invention are used to identify individuals with hepatoma when screening candidates for a clinical study; such individuals can then be included in or excluded from the study or can be placed in a separate cohort for treatment or analysis. If desired, the candidates can concurrently be screened to identify individuals with Hepatitis B and/or Hepatitis C; procedures for these screens are well known in the art and include, for instance, serological studies to detect antibodies to one or more Hepatitis B or Hepatitis C antigens, and PCR studies to identify one or more oligonucleotide sequences from the Hepatitis B or Hepatitis C genome.

### 5.5. Purification of HPIs

In particular aspects, the invention provides isolated HPIs, preferably human HPIs, and fragments and derivatives thereof which comprise an antigenic determinant (i.e., can be recognized by an antibody) or which are otherwise functionally active, as well as nucleic acid sequences encoding the foregoing. “Functionally active” HPI as used herein refers to that material displaying one or more known functional activities associated with a full-length (wild-type) HPI, e.g., binding to an HPI substrate or HPI binding partner, antigenicity (binding to an anti-target antibody), immunogenicity, etc.

In specific embodiments, the invention provides fragments of an HPI comprising at least 6 amino acids, 10 amino acids, 50 amino acids, or at least 75 amino acids. Fragments, or proteins comprising fragments, lacking some or all of the regions of an HPI are also provided. Nucleic acids encoding the foregoing are provided.

Once a recombinant nucleic acid which expresses the HPI gene sequence is identified, the gene product can be analyzed. This is achieved by assays based on the physical or functional properties of the product, including radioactive labelling of the product followed by analysis by gel electrophoresis, immunoassay, etc.

Once the HPI is identified, it can be isolated and purified by standard methods including chromatography (e.g., ion exchange, affinity, and sizing column chromatography), centrifugation, differential solubility, or by any other standard technique for the purification of proteins.

Alternatively, once an HPI produced by a recombinant nucleic acid is identified, the entire amino acid sequence of the HPI can be deduced from the nucleotide sequence of the chimeric gene contained in the recombinant. As a result, the protein can be synthesized by standard chemical methods known in the art (e.g., see Hunkapiller, et al., 1984, Nature 310:105-111).

In another alternative embodiment, native HPIs can be purified from natural sources, by standard methods such as those described above (e.g., immunoaffinity purification).

In a preferred embodiment, HPIs are isolated by the Preferred Technology described in U.S. application Ser. No. 08/980,574 and WO 98/23950 which are incorporated herein by reference. For preparative-scale runs, a narrow-range “zoom gel” having a pH range of 2 pH units or less is preferred for the isoelectric focusing step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated HPI that can be recovered from the gel. When used in this way for preparative-scale runs, the Preferred Technology typically provides up to 100 ng, and can provide up to 1000 ng, of an isolated HPI in a single run. Those of skill in the art will appreciate that a zoom gel can be used in any separation strategy which employs gel isoelectric focusing.

In a specific embodiment of the present invention, such HPIs, whether produced by recombinant DNA techniques or by chemical synthetic methods or by purification of native proteins, include (but are not limited to) those containing, as a primary amino acid sequence, all or part of the amino acid sequence of the HPI, as well as fragments and other derivatives, and analogs thereof, including proteins homologous thereto.

### 5.6. Production of Antibodies to HPIs

According to the invention, an HPI, its fragments or other derivatives, or analogs thereof, may be used as an immunogen to generate antibodies which immunospecifically bind such an immunogen. Such proteins, fragments, derivatives, or analogs can be isolated by any convenient means, including the methods described in the preceding section of this application. The antibodies generated include but are not limited to polyclonal, monoclonal, chimeric, single chain, Fab fragments, and an Fab expression library. In a specific embodiment, antibodies to a human HPI are produced. In another embodiment, antibodies to a domain of an HPI are produced. In a specific embodiment, hydrophilic fragments of an HPI are used as immunogens for antibody production.

Polyclonal antibodies which may be used in the methods of the invention are heterogeneous populations of antibody molecules derived from the sera of immunized animals. Various procedures known in the art may be used for the production of polyclonal antibodies to an HPI or derivative or analog. In a particular embodiment, rabbit polyclonal antibodies to an epitope of an HPI, or a subsequence thereof, can be obtained. For the production of antibody, various host animals can be immunized by injection with the native HPI, or a synthetic version, or derivative (e.g., fragment) thereof, including but not limited to rabbits, mice, rats, horses, goats, chickens, etc. Various adjuvants may be used to increase the immunological response, depending on the host species, and including but not limited to complete or incomplete Freund's adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward an HPI sequence or analog thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). (Each of the foregoing references in incorporated herein by reference.) In an additional embodiment of the invention, monoclonal antibodies can be produced in germ-free animals as described in PCT/US90/02545, which is incorporated herein by reference. According to the invention, human antibodies may be used and can be obtained by using human hybridomas (Cote et al., 1983, Proc. Natl. Acad. Sci. USA 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. USA 81:6851-6855; Neuberger et al., 1984, Nature 312:604-608; Takeda et al., 1985, Nature 314:452-454) by splicing the genes from a mouse antibody molecule specific for an HPI together with genes from a human antibody molecule of appropriate biological activity can be used; such antibodies are within the scope of this invention. (Each of the foregoing references is incorporated herein by reference.)

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with selected antigens, e.g., all or a portion of a HPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Biotechnology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of suitable techniques which can be used to produce single-chain Fvs and antibodies against HPIs of the present invention include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fuised to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology,1986, 121:210.

According to the invention, techniques described for the production of single chain antibodies (U.S. Pat. No. 4,946,778, incorporated herein by reference) can be adapted to produce HPI-specific single-chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for HPIs, derivatives, or analogs.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical or unnatural amino acids.

In the production of antibodies, screening for the desired antibody can be accomplished by techniques known in the art, e.g., ELISA (enzyme-linked imrunosorbent assay). For example, to select antibodies which recognize a specific domain of an HPI, one may assay generated hybridomas for a product which binds to an HPI fragment containing such a domain. For selection of an antibody that specifically binds a first HPI homolog but which does not specifically bind a different HPI homolog, one can select on the basis of positive binding to the first HPI homolog and a lack of binding to the second HPI homolog. Similarly, for selection of an antibody that specifically binds an HPI but which does not specifically bind a different isoform of the same protein (e.g., a different glycoform having the same core peptide as the HPI), one can select on the basis of positive binding to the HPI and a lack of binding to the different isoform (e.g., glycoform).

Antibodies specific to a domain of an HPI are also provided.

The foregoing antibodies can be used in methods known in the art relating to the localization and activity of the HPIs of the invention, e.g., for imaging these proteins, measuring levels thereof in appropriate physiological samples, in diagnostic methods, etc.

### 5.7. Expression of Antibodies

The antibodies of the invention can be produced by any suitable method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulihydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen based on the present description which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, an increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+nitriloacetic acid-agarose columns and histidine-tagged proteins are selectively eluted with imidazole-containing buffers.

### 5.8. Conjugated Antibodies

In a preferred embodiment, anti-HPI antibodies or fragments thereof are conjugated to a diagnostic or a therapeutic moiety. The antibodies can be used, for example, for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

Anti-HPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker. Inc. 1987); Thorpe, “Antibody Carriers of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective of The Therapeutic Use of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties of Antibody-Toxin Conjugates”, Immunol. Rev., 62:119-58 (1982). These references are incorporated herein in their entirety.

Alternatively, an antibody can be conjugated to a second antibody to form an antibody heteroconjugate as described in U.S. Pat. No. 4,676,980.

An antibody with or without a therapeutic moiety conjugated to it can be used as a therapeutic that is administered alone or in combination with cytotoxic factor(s) and/or cytokine(s).

### 5.9. Isolation Of DNA Encoding An HPI

Specific embodiments for the cloning of an HPI gene, are presented below by way of example and not of limitation.

The nucleotide sequences of the present invention, including DNA and RNA, and comprising a sequence encoding the HPI or a fragment or analog thereof, may be synthesized using methods known in the art, such as using conventional chemical approaches or polymerase chain reaction (PCR) amplification of overlapping oligonucleotides. The sequences also provide for the identification and cloning of the HPI gene from any species, for instance for screening cDNA libraries, genomic libraries or expression libraries.

For example, to clone a gene encoding a HPI by PCR techniques, anchored degenerate oligonucleotides (or a set of most likely oligonucleotides) can be designed for all HPI peptide fragments identified as part of the same protein. PCR reactions under a variety of conditions can be performed with relevant cDNA and genomic DNAs (e.g., from tissue homogenates or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer KD, Biotechniques, 1995, 19(4):550-2). Vectorette PCR may be performed with probes that are, for example, anchored degenerate oligonucleotides (or most likely oligonucleotides) coding for HPI peptide fragments, using as a template a genomic library or cDNA library pools.

Anchored degenerate oligonucleotides (and most likely oligonucleotides) can be designed for all HPI peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

The nucleotide sequences comprising a sequence encoding an HPI of the present invention are useful for their ability to selectively form duplex molecules with complementary stretches of other protein genes. Depending on the application, a variety of hybridization conditions may be employed to achieve varying sequence identities.

For a high degree of selectivity, relatively stringent conditions are used to form the duplexes, such as low salt or high temperature conditions. As used herein, “highly stringent conditions” means hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3; incorporated herein by reference in its entirety.) For some applications, less stringent hybridization conditions are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of fornamide, to destabilize the hybrid duplex. Thus, particular hybridization conditions can be readily manipulated, and will generally be chosen depending on the desired results. For example, convenient hybridization temperatures in the presence of 50% formamide are: 42° C. for a probe which is 95 to 100% homologous to the HPI gene fragment, 37° C. for 90 to 95% homology and 32° C. for 70 to 90% homology.

In the preparation of genomic libraries, DNA fragments are generated, some of which will encode a part or the whole of an HPI. The DNA may be cleaved at specific sites using various restriction enzymes. Alternatively, one may use DNase in the presence of manganese to fragment the DNA, or the DNA can be physically sheared, as for example, by sonication. The DNA fragments can then be separated according to size by standard techniques, including but not limited to, agarose and polyacrylamide gel electrophoresis, column chromatography and sucrose gradient centrifugation. The DNA fragments can then be inserted into suitable vectors, including but not limited to plasmids, cosmids, bacteriophages lambda or T₄, and yeast artificial chromosome (YAC). (See, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. USA 72:3961).

The genomic libraries may be screened with labeled degenerate oligonucleotide probes corresponding to the amino acid sequence of any peptide of the HPI using optimal approaches well known in the art. Any probe used preferably is at least 10 nucleotides (more preferably 15 nucleotides, still more preferably 20 nucleotides) in length.

As shown in Tables III and IV above, some HPIs disclosed herein correspond to previously identified proteins encoded by genes whose sequences are publicly known. To screen such a gene, any probe may be used that is complementary to the gene or its complement; preferably the probe is 10 nucleotides or longer, more preferably 15 nucleotides or longer. The Entrez database held by the National Center for Biotechnology Information (NCBI)—which is accessible at http://www.ncbi.nlm.nih.gov/—(and, for HPI-16, the NCBI non-redundant database) provides gene sequences for these HPIs under the following accession numbers, and each sequence is incorporated herein by reference:

TABLE V
Gene sequences of HPI-related proteins

| HF # | HPI | Accession numbers |
|---|---|---|
| 1 | HPI-1 | AA617854, AA630254, AA580356, AA715907, |
| | | AA614684, AA580429, AA523377 |
| 17 | HPI-2 | U24578, M59815, M59816, K02403, AF019413 |
| 18 | HPI-3 | T40940, T40002 |
| 20 | HPI-4 | T19152, H73939, T40182, T40167, T40158, Z20894 |
| 21 | HPI-5 | T19152, H73939, T40182, T40167, T40158, Z20894 |
| 23 | HPI-6 | U24578, M59815, M59816, K02403, AF019413 |
| 25 | HPI-8 | U24578, M59815, M59816, K02403, AF019413 |
| 28 | HPI-9 | T41010, T40102, T40058, T39954 |
| 29 | HPI-10 | T41056, T40108, Z21017, Z20888, Z19984, Z19971 |
| 29 | HPI-11 | T40178, Z21027, Z19988 |
| 31 | HPI-12 | T19152, H73939, T40182, T40167, T40158, Z20894 |
| 33 | HPI-14 | U24578, M59815, M59816, K02403, AF019413 |
| 34 | HPI-15 | U24578, M59815, M59816, K02403, AF019413 |
| 36 | HPI-16 | NCBI non-redundant database published Nov. 22, 1997, |
| | | Accession # 1082547 |
| 39 | HPI-17 | U24578, M59815, M59816, K02403, AF019413 |
| 44 | HPI-18 | U24578, M59815, M59816, K02403, AF019413 |
| 51 | HPI-19 | AA269874 |
| 52 | HPI-20 | AA269874 |
| 55 | HPI-22 | AA269874 |

When no nucleotide sequence is known that encodes a given HPI, degenerate probes can be used for screening. In Table VI, a degenerate set of probes is provided for each of the following HPIs: HPI-13 and HPI-21. In the method used for sequencing by mass spectroscopy in the present invention, the following sets of amino acids cannot be distinguished since they have the same mass: leucine (L) and isoleucine (I); asparagine (N) and two glycines (GG). Furthermore, the mass accuracy of the tandem mass spectrometer used for amino acid sequencing in the method of the present invention was insufficient to distinguish between the following sets of amino acids: phenylalanine (F) and oxidized methionine (M*); tryptophan (W) and the combination of aspartic acid and alanine (i.e. DA or AD); tryptophan (W) and the combination of glutamic acid (E) and glycine (G) (i.e. EG or GE); tryptophan (W) and the combination of valine (V) and serine (S) (i.e. VS or SV). In Table VI, each possible amino acid sequence is listed for each sequence determined by mass spectroscopy, and preferred and fully degenerate sets of probes for each possible amino acid sequence are provided.

TABLE VI
Amino Acid Sequences and Probes for HPIs

| | | Partial Amino Acid | | | | | |
|---|---|---|---|---|---|---|---|
| | | Sequence as | | | | | |
| | | Determined by | | | | | |
| HF# | HPI# | Mass Spectrometry | | Preferred Probes | | Degenerate Probes | |
| HF-31 | HPI-13 | AETTDF | (SEQ ID NO:22) | GCCGAGACCACCGACTTC | (SEQ ID NO:23) | GCNGARACNACNG | (SEQ ID NO:24) |
| | | | | | | AYTTY | |
| HF-31 | HPI-13 | AETTDM | (SEQ ID NO:25) | GCCGAGACCACCGACATG | (SEQ ID NO:26) | GCNGARACNACNG | (SEQ ID NO:27) |
| | | | | | | AYATG | |
| HF-54 | HPI-21 | LWLGTTR | (SEQ ID NO:28) | CTGTGGCTGGGCACCACCC | (SEQ ID NO:29) | YTNTGGYTNGGNA | (SEQ ID NO:30) |
| | | | | GC | | CNACNMGN | |
| HF-54 | HPI-21 | LDALGTTR | (SEQ ID NO:31) | CTGGACGCCCTGGGCACCA | (SEQ ID NO:32) | YTNGAYGCNYTNG | (SEQ ID NO:33) |
| | | | | CCCGC | | GNACNACNMGN | |
| HF-54 | HPI-21 | LADLGTTR | (SEQ ID NO:34) | CTGGCCGACCTGGGCACCA | (SEQ ID NO:35) | YTNGCNGAYYTNG | (SEQ ID NO:36) |
| | | | | CCCGC | | GNACNACNMGN | |
| HF-54 | HPI-21 | LGELGTTR | (SEQ ID NO:37) | CTGGGCGAGCTGGGCACCA | (SEQ ID NO:38) | YTNGGNGARYTNG | (SEQ ID NO:39) |
| | | | | CCCGC | | GNACNACNMGN | |
| HF-54 | HPI-21 | LEGLGTTR | (SEQ ID NO:40) | CTGGAGGCCCTGGGCACCA | (SEQ ID NO:41) | YTNGARGGNYTNG | (SEQ ID NO:42) |
| | | | | CCCGC | | GNACNACNMGN | |
| HF-54 | HPI-21 | LVSLGTTR | (SEQ ID NO:43) | CTGGTGAGCCTGGGCACCA | (SEQ ID NO:44) | YTNGTNWSNYTNG | (SEQ ID NO:45) |
| | | | | CCCGC | | GNACNACNMGN | |
| HF-54 | HPI-21 | LSVLGTTR | (SEQ ID NO:46) | CTGAGCGTGCTGGGCACCA | (SEQ ID NO:47) | YTNWSNGTNYTNG | (SEQ ID NO:48) |
| | | | | CCCGC | | GNACNACNMGN | |
| HF-54 | HPI-21 | IWLGTTR | (SEQ ID NO:49) | ATCTGGCTGGGCACCACCC | (SEQ ID NO:50) | ATHTGGYTNGGNA | (SEQ ID NO:51) |
| | | | | GC | | CNACNMGN | |
| HF-54 | HPI-21 | IDALGTTR | (SEQ ID NO:52) | ATCGACGCCCTGGGCACCA | (SEQ ID NO:53) | ATHGAYGCNYTNG | (SEQ ID NO:54) |
| | | | | CCCGC | | GNACNACNMGN | |
| HF-54 | HPI-21 | IADLGTTR | (SEQ ID NO:55) | ATCGCCGACCTGGGCACCA | (SEQ ID NO:56) | ATHGCNGAYYTNG | (SEQ ID NO:57) |
| | | | | CCCGC | | GNACNACNMGN | |
| HF-54 | HPI-21 | IGELGTTR | (SEQ ID NO:58) | ATCGGCGAGCTGGGCACCA | (SEQ ID NO:59) | ATHGGNGARYTNG | (SEQ ID NO:60) |
| | | | | CCCGC | | GNACNACNMGN | |
| HF-54 | HPI-21 | IEGLGTTR | (SEQ ID NO:61) | ATCGAGGCCCTGGGCACCA | (SEQ ID NO:62) | ATHGARGGNYTNG | (SEQ ID NO:63) |
| | | | | CCCGC | | GNACNACNMGN | |
| HF-54 | HPI-21 | IVSLGTTR | (SEQ ID NO:64) | ATCGTGAGCCTGGGCACCA | (SEQ ID NO:65) | ATHGTNWSNYTNG | (SEQ ID NO:66) |
| | | | | CCCGC | | GNACNACNMGN | |
| HF-54 | HPI-21 | ISVLGTTR | (SEQ ID NO:67) | ATCAGCGTGCTGGGCACCA | (SEQ ID NO:68) | ATHWSNGTNYTNG | (SEQ ID NO:69) |
| | | | | CCCGC | | GNACNACNMGN | |
| HF-54 | HPI-21 | LWIGTTR | (SEQ ID NO:70) | CTGTGGATCGGCACCACCC | (SEQ ID NO:71) | YTNTGGATHGGNA | (SEQ ID NO:72) |
| | | | | GC | | CNACNMGN | |
| HF-54 | HPI-21 | LDAIGTTR | (SEQ ID NO:73) | CTGGACGCCATCGGCACCA | (SEQ ID NO:74) | YTNGAYGCNATHG | (SEQ ID NO:75) |
| | | | | CCCGC | | GNACNACNMGN | |
| HF-54 | HPI-21 | LADIGTTR | (SEQ ID NO:76) | CTGGCCGACATCGGCACCA | (SEQ ID NO:77) | YTNGCNGAYATHG | (SEQ ID NO:78) |
| | | | | CCCGC | | GNACNACNMGN | |
| HF-54 | HPI-21 | LGEIGTTR | (SEQ ID NO:79) | CTGGGCGAGATCGGCACCA | (SEQ ID NO:80) | YTNGGNGARATHG | (SEQ ID NO:81) |
| | | | | CCCGC | | GNACNACNMGN | |
| HF-54 | HPI-21 | LEGIGTTR | (SEQ ID NO:82) | CTGGAGGCCATCGGCACCA | (SEQ ID NO:83) | YTNGARGGNATHG | (SEQ ID NO:84) |
| | | | | CCCGC | | GNACNACNMGN | |
| HF-54 | HPI-21 | LVSIGTTR | (SEQ ID NO:85) | CTGGTGAGCATCGGCACCA | (SEQ ID NO:86) | YTNGTNWSNATHG | (SEQ ID NO:87) |
| | | | | CCCGC | | GNACNACNMGN | |
| HF-54 | HPI-21 | LSVIGTTR | (SEQ ID NO:88) | CTGAGCGTGATCGGCACCA | (SEQ ID NO:89) | YTNWSNGTNATHG | (SEQ ID NO:90) |
| | | | | CCCGC | | GNACNACNMGN | |
| HF-54 | HPI-21 | IWIGTTR | (SEQ ID NO:91) | CTGTGGATCGGCACCACCC | (SEQ ID NO:92) | ATHTGGATHGGNA | (SEQ ID NO:93) |
| | | | | GC | | CNACNMGN | |
| HF-54 | HPI-21 | IDAIGTTR | (SEQ ID NO:94) | ATCGACGCCATCGGCACCA | (SEQ ID NO:95) | ATHGAYGCNATHG | (SEQ ID NO:96) |
| | | | | CCCGC | | GNACNACNMGN | |
| HF-54 | HPI-21 | IADIGTTR | (SEQ ID NO:97) | ATCGCCGACATCGGCACCA | (SEQ ID NO:98) | ATHGCNGAYATHG | (SEQ ID NO:99) |
| | | | | CCCGC | | GNACNACNMGN | |
| HF-54 | HPI-21 | IGEIGTTR | (SEQ ID NO:100) | ATCGGCGAGATCGGCACCA | (SEQ ID NO:101) | ATHGGNGARATHG | (SEQ ID NO:102) |
| | | | | CCCGC | | GNACNACNMGN | |
| HF-54 | HPI-21 | IEGIGTTR | (SEQ ID NO:103) | ATCGAGGCCATCGGCACCA | (SEQ ID NO:104) | ATHGARGGNATHG | (SEQ ID NO:105) |
| | | | | CCCGC | | GNACNACNMGN | |
| HF-54 | HPI-21 | IVSIGTTR | (SEQ ID NO:106) | ATCGTGAGCATCGGCACCA | (SEQ ID NO:107) | ATHGTNWSNATHG | (SEQ ID NO:108) |
| | | | | CCCGC | | GNACNACNMGN | |
| HF-54 | HPI-21 | ISVIGTTR | (SEQ ID NO:109) | ATCAGCGTGATCGGCACCA | (SEQ ID NO:110) | ATHWSNGTNATHG | (SEQ ID NO:111) |
| | | | | CCCGC | | GNACNACNMGN | |

In Table VI, supra, the preferred and degenerate sets of probes are described using GCG Nucleotide Ambiguity Codes as employed in GCG SeqWeb™ sequence analysis software (SeqWeb™ version 1.1, part of Wisconsin Package Version 10, Genetics Computer Group, Inc.). These Nucleotide Ambiguity Codes have the following meaning:

| GCG Code | Meaning |
|---|---|
| A | A |
| C | C |
| G | G |
| T | T |
| U | T |
| M | A or C |
| R | A or G |
| W | A or T |
| S | C or G |
| Y | C or T |
| K | G or T |
| V | A or C or G |
| H | A or C or T |
| D | A or G or T |
| B | C or G or T |
| X | G or A or T or C |
| N | G or A or T or C |

GCG uses the letter codes for amino acid codes and nucleotide ambiguity proposed by IUPAC-IUB. These codes are compatible with the codes used by the EMBL, GenBank, and PIR databases. See IUPAC, Commission on Nomenclature of Organic Chemistry. A Guide to IUPAC Nomenclature of Organic Compounds (Recommendations 1993), Blackwell Scientific publications, 1993.

Clones in libraries with insert DNA encoding the HPI or fragments thereof will hybridize to one or more of the degenerate oligonucleotide probes (or their complement). Hybridization of such oligonucleotide probes to genomic libraries are carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the same conditions.

In yet another aspect, clones of nucleotide sequences encoding a part or the entire HPI or HPI-derived polypeptides may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed HPI or HPI-derived polypeptides. In one embodiment, the various anti-HPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Clones or plaques from the library are brought into contact with the antibodies to identify those clones that bind.

In an embodiment, colonies or plaques containing DNA that encodes an HPI or HPI-derived polypeptide can be detected using DYNA Beads according to Olsvick et al., 29th ICAAC, Houston, Tex. 1989, incorporated herein by reference. Anti-HPI antibodies are crosslinked to tosylated DYNA Beads M280, and these antibody-containing beads would then be used to adsorb to colonies or plaques expressing HPI or HPI-derived polypeptide. Colonies or plaques expressing an HPI or HPI-derived polypeptide are identified as any of those that bind the beads.

Alternatively, the anti-HPI antibodies can be nonspecifically immobilized to a suitable support, such as silica or Celite™ resin. This material would then be used to adsorb to bacterial colonies expressing the HPI protein or HPI-derived polypeptide as described in the preceding paragraph.

In another aspect, PCR amplification may be used to produce substantially pure DNA encoding a part of or the whole of an HPI from genomic DNA. Oligonucleotide primers, degenerate or otherwise, corresponding to known HPI sequences can be used as primers.

PCR can be carried out, e.g., by use of a Perkin-Elmer Cetus thermal cycler and a thermostable DNA polymerase such as Thermus aquaticus DNA polymerase (Gene Amp™ or AmpliTaq™ DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding an HPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The HPI gene can also be identified by mRNA selection by nucleic acid hybridization followed by in vitro translation. In this procedure, fragments are used to isolate complementary mRNAs by hybridization. Such DNA fragments may represent available, purified HPI DNA of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against an HPI. A radiolabelled HPI cDNA can be synthesized using the selected MRNA (from the adsorbed polysomes) as a template. The radiolabelled mRNA or cDNA may then be used as a probe to identify the HPI DNA fragments from among other genomic DNA fragments.

Alternatives to isolating HPI genomic DNA include, but are not limited to, chemically synthesizing the gene sequence itself from a known sequence or making CDNA to the mRNA which encodes the HPI. For example, RNA for cDNA cloning of the HPI gene can be isolated from cells which express the HPI. Other methods are possible and within the scope of the invention.

Any eukaryotic cell potentially can serve as the nucleic acid source for the molecular cloning of the HPI gene. The nucleic acid sequences encoding the HPI can be isolated from vertebrate, mammalian, human, porcine, bovine, feline, avian, equine, canine, as well as additional primate sources, insects, plants, etc. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences. Whatever the source, the HPI gene should be molecularly cloned into a suitable vector for propagation.

The identified and isolated gene or cDNA can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives or the Bluescript vector (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and HPI gene may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated HPI gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The HPI sequences provided by the instant invention include those nucleotide sequences encoding substantially the same amino acid sequences as found in native HPIs, and those encoded amino acid sequences with functionally equivalent amino acids, as well as those encoding other target derivatives or analogs.

In a specific embodiment, an isolated nucleic acid molecule encoding a HPI-related polypeptide can be created by introducing one or more nucleotide substitutions, additions or deletions into the nucleotide sequence of a HPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

### 5.10 Expression of DNA Encoding HPIs

The nucleotide sequence encoding an HPI or a functionally active analog or fragment or other derivative thereof can be inserted into an appropriate expression vector, i.e., a vector which contains the necessary elements for the transcription and translation of the inserted protein-coding sequence. The necessary transcriptional and translational signals can also be supplied by the native HPI gene or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, the human HPI gene is expressed, or a sequence encoding a functionally active portion of the human HPI. In yet another embodiment, a fragment of target comprising a domain of the HPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an HPI or peptide fragment may be regulated by a second nucleic acid sequence so that the HPI or peptide is expressed in a host transformed with the recombinant DNA molecule. For example, expression of an HPI gene may be controlled by any promoter or enhancer element known in the art. Promoters which may be used to control HPI gene expression include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. USA 75:3727-3731), or the tac promoter (DeBoer, et al., 1983, Proc. Natl. Acad. Sci. USA 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

In a specific embodiment, a vector is used that comprises a promoter operably linked to an HPI-encoding nucleic acid, one or more origins of replication, and, optionally, one or more selectable markers (e.g., an antibiotic resistance gene).

In a specific embodiment, an expression construct is made by subcloning an HPI coding sequence into the EcoRI restriction site of each of the three pGEX vectors (Glutathione S-Transferase expression vectors; Smith and Johnson, 1988, Gene 7:31-40). This allows for the expression of the HPI product from the subclone in the correct reading frame.

In mammalian host cells, a number of viral-based expression systems may be utilized. In cases where an adenovirus is used as an expression vector, the HPI coding sequence or HPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing HPI gene inserts can be identified by three general approaches: (a) nucleic acid hybridization, (b) presence or absence of “marker” gene functions, and (c) expression of inserted sequences. In the first approach, the presence of an HPI gene inserted in an expression vector can be detected by nucleic acid hybridization using probes comprising sequences that are homologous to an inserted HPI gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of an HPI gene in the vector. For example, if the HPI gene is inserted within the marker gene sequence of the vector, recombinants containing the HPI gene insert can be identified by the absence of the marker gene function. In the third approach, recombinant expression vectors can be identified by assaying the HPI gene product expressed by the recombinant. Such assays can be based, for example, on the physical or functional properties of the HPI gene product in in vitro assay systems, e.g., binding with anti-HPI antibody.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered HPI may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may affect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other specific embodiments, the HPI, fragment, analog, or derivative may be expressed as a fusion, or chimeric protein product (comprising the protein, fragment, analog, or derivative joined via a peptide bond to a heterologous protein sequence (of a different protein)). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding a HPI, a fragment of a HPI, a HPI-related polypeptide, or a fragment of a HPI-related polypeptide can be fused to an epitope tag (e.g., the hemagglutinin (“HA”) tag or flag tag) to aid in detection and purification of the expressed polypeptide. For example, a system described by Jankiiecht et al. allows for the ready purification of non-denatured fusion proteins expressed in human cell lines (Janknecht et al., 1991, Proc. Natl. Acad. Sci. USA 88:8972-897).

Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

Both cDNA and genomic sequences can be cloned and expressed.

### 5.11 Domain Structure of HPIs

Domains of some of the HPIs provided by the present invention are known in the art and have been described in the scientific literature. Moreover, domains of a HPI can be identified using techniques known to those of skill in the art. For example, one or more domains of a HPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347, 166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, those skilled in the art can identify domains of a HPI having enzymatic or binding activity, and further can identify nucleotide sequences encoding such domains. These nucleotide sequences can then be used for recombinant expression of a HPI fragment that retains the enzymatic or binding activity of the HPI.

Based on the present description, those skilled in the art can identify domains of a HPI having enzymatic or binding activity, and further can identify nucleotide sequences encoding such domains. These nucleotide sequences can then be used for recombinant expression of HPI fragments that retain the enzymatic or binding activity of the HPI.

In one embodiment, a HPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A HPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay.

### 5.12 Diagnosis of Hepatoma

In accordance with the present invention, suitable test samples, e.g., of serum or tissue, obtained from a subject suspected of having or known to have hepatoma can be used for diagnosis. In one embodiment, a decreased abundance of one or more HFs or HPIs (or any combination of them) in a first test sample relative to a second control sample (from a subject or subjects free from hepatoma) or a previously determined reference range indicates the presence of hepatoma; HFs and HPIs suitable for this purpose are identified in Tables I and III, respectively, as described in detail above. In another embodiment of the invention, an increased abundance of one or more HFs or HPIs (or any combination of them) in a first sample compared to a second sample or a previously determined reference range indicates the presence of hepatoma; HFs and HPIs suitable for this purpose are identified in Tables II and IV, respectively, as described in detail above. In another embodiment, the relative abundance of one or more HFs or HPIs (or any combination of them) in a test sample compared to a control sample or a previously determined reference range indicates a subtype of hepatoma (e.g., primary or metastatic hepatoma). In yet another embodiment, the relative abundance of one or more HFs or HPIs (or any combination of them) in a test sample relative to a control sample or a previously determined reference range indicates the degree or severity of hepatoma. In any of the aforesaid methods, detection of one or more HPIs described herein may optionally be combined with detection of one or more additional biomarkers for hepatoma including, but not limited to AFP. Any suitable method in the art can be employed to measure the level of HFs and HPIs, including but not limited to the Preferred Technology described herein, kinase assays, immunoassays to detect and/or visualize the HPIs (e.g., Western blot, immunoprecipitation followed by sodium dodecyl sulfate polyacrylamide gel electrophoresis, immunocytochemistry, etc.). In cases where a HPI has a known function, an assay for that function may be used to measure HPI expression. In a further embodiment, a decreased abundance of mRNA encoding one or more HPIs identified in Table III (or any combination of them) in a first test sample relative to a second control sample or a previously determined reference range indicates the presence of hepatoma. In yet a further embodiment, an increased abundance of MRNA encoding one or more HPIs identified in Table IV (or any combination of them) in a first test sample relative to a second control sample or previously determined reference range indicates the presence of hepatoma. Any suitable hybridization assay can be used to detect HPI expression by detecting and/or visualizing mRNA encoding the HPI (e.g., Northern assays, dot blots, in situ hybridization, etc.).

In another embodiment of the invention, labeled antibodies, derivatives and analogs thereof, which specifically bind to a HPI can be used for diagnostic purposes, e.g., to detect, diagnose, or monitor hepatoma. Preferably, hepatoma is detected in an animal, more preferably in a mammal and most preferably in a human.

### 5.13 Screening Assays

The invention provides methods for identifying agents (e.g., chemical compounds, proteins, or peptides) that bind to a HPI or have a stimulatory or inhibitory effect on the expression or activity of a HPI. The invention also provides methods of identifying candidate agents that bind to a HPI-related polypeptide or a HPI fusion protein or have a stimulatory or inhibitory effect on the expression or activity of a HPI-related polypeptide or a HPI fusion protein. Examples of candidate agents include, but are not limited to, nucleic acids (e.g., DNA and RNA), carbohydrates, lipids, proteins, peptides, peptidomimetics, small molecules, agonists, antagonists and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. Nos. 5,738,996; and 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, candidate agents that interact with (i.e., bind to) a HPI, a HPI fragment (e.g. a functionally active fragment), a HPI-related polypeptide, a fragment of a HPI-related polypeptide, or a HPI fusion protein are identified in a cell-based assay system. In accordance with this embodiment, cells expressing a HPI, a fragment of a HPI, a HPI-related polypeptide, a fragment of a HPI-related polypeptide, or a HPI fusion protein are contacted with a candidate agent or a control agent and the ability of the candidate agent to interact with the HPI is determined. If desired, this assay may be used to screen a plurality (e.g. a library) of candidate agents. The cell, for example, can be of prokaryotic origin (e.g., E. coli) or eukaryotic origin (e.g., yeast or mammalian). Further, the cells can express the HPI, fragment of the HPI, HPI-related polypeptide, a fragment of the HPI-related polypeptide, or a HPI fusion protein endogenously or be genetically engineered to express the HPI, fragment of the HPI, HPI-related polypeptide, a fragment of the HPI-related polypeptide, or a HPI fusion protein. In some embodiments, the HPI, fragment of the HPI, HPI-related polypeptide, a fragment of the HPI-related polypeptide, or a HPI fusion protein or the candidate agent is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a HPI and a candidate agent. The ability of the candidate agent to interact directly or indirectly with a HPI, a fragment of a HPI, a HPI-related polypeptide, a fragment of a HPI-related polypeptide, or a HPI fusion protein can be determined by methods known to those of skill in the art. For example, the interaction between a candidate agent and a HPI, a fragment of a HPI, a HPI-related polypeptide, a fragment of a HPI-related polypeptide, or a HPI fusion protein can be determined by flow cytometry, a scintillation assay, immunoprecipitation or western blot analysis.

In another embodiment, agents that interact with (i.e., bind to) a HPI, a HPI fragment (e.g., a functionally active fragment) a HPI-related polypeptide, a fragment of a HPI-related polypeptide, or a HPI fusion protein are identified in a cell-free assay system. In accordance with this embodiment, a native or recombinant HPI or fragment thereof, or a native or recombinant HPI-related polypeptide or fragment thereof, or a HPI-fusion protein or fragment thereof, is contacted with a candidate agent or a control agent and the ability of the candidate agent to interact with the HPI or HPI-related polypeptide, or HPI fusion protein is determined. If desired, this assay may be used to screen a plurality (e.g. a library) of candidate agents. Preferably, the HPI, HPI fragment, HPI-related polypeptide, fragment of a HPI-related polypeptide, or HPI-fusion protein is first immobilized, by, for example, contacting the HPI, HPI fragment, HPI-related polypeptide, fragment of a HPI-related polypeptide, or HPI fusion protein with an immobilized antibody which specifically recognizes and binds it, or by contacting a purified preparation of the HPI, HPI fragment, HPI-related polypeptide, fragment of a HPI-related polypeptide, or HPI fusion protein with a surface designed to bind proteins. The HPI, HPI fragment, HPI-related polypeptide, fragment of a HPI-related polypeptide, or HPI fusion protein may be partially or completely purified (e.g., partially or completely free of other polypeptides) or part of a cell lysate. Further, the HPI, HPI fragment, HPI-related polypeptide, fragment of a HPI-related polypeptide may be a fusion protein comprising the HPI or a biologically active portion thereof, or HPI-related polypeptide and a domain such as glutathionine-S-transferase. Alternatively, the HPI, HPI fragment, HPI-related polypeptide, fragment of a HPI-related polypeptide or HPI fusion protein can be biotinylated using techniques well known to those of skill in the art (e.g., biotinylation kit, Pierce Chemicals; Rockford, Ill.). The ability of the candidate compound to interact with a HPI, HPI fragment, HPI-related polypeptide, fragment of a HPI-related polypeptide, or HPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a HPI or is responsible for the post-translational modification of a HPI. In a primary screen, a plurality (e.g., a library) of agents are contacted with cells that naturally or recombinantly express: (i) a HPI, an isoform of a HPI, a HPI homologue, a HPI-related polypeptide, a HPI fusion protein, or a biologically active fragment of any of the foregoing; and (ii) a protein that is responsible for processing of the HPI, HPI isoform, HPI homologue, HPI-related polypeptide, HPI fusion protein, or fragment in order to identify compounds that modulate the production, degradation, or post-translational modification of the HPI, HPI isoform, HPI homologue, HPI-related polypeptide, HPI fusion protein or fragment. If desired, compounds identified in the primary screen can then be assayed in a secondary screen against cells naturally or recombinantly expressing the specific HPIs of interest. The ability of the candidate agent to modulate the production, degradation or post-translational modification of a HPI, isoform, homologue, HPI-related polypeptide, or HPI fusion protein can be determined by methods known to those of skill in the art, including without limitation, flow cytometry, a scintillation assay, immunoprecipitation and western blot analysis.

In another embodiment, agents that competitively interact with (i.e., bind to) a HPI, HPI fragment, HPI-related polypeptide, a fragment of a HPI-related polypeptide, or a HPI fusion protein are identified in a competitive binding assay. In accordance with this embodiment, cells expressing a HPI, HPI fragment, HPI-related polypeptide, a fragment of a HPI-related polypeptide, or a HPI fusion protein are contacted with a candidate agent and an agent known to interact with the HPI, HPI fragment, HPI-related polypeptide, a fragment of a HPI-related polypeptide or a HPI fusion protein; the ability of the candidate agent to competitively interact with the HPI, HPI fragment, HPI-related polypeptide, fragment of a HPI-related polypeptide, or HPI fusion protein is then determined. Alternatively, agents that competitively interact with (i.e., bind to) a HPI, HPI fragment, HPI-related polypeptide or fragment of a HPI-related polypeptide are identified in a cell-free assay system by contacting a HPI, HPI fragment, HPI-related polypeptide, fragment of a HPI-related polypeptide, or a HPI fusion protein with a candidate agent and a control agent known to interact with the HPI, HPI-related polypeptide or HPI fusion protein. As stated above, the ability of the candidate agent to interact with a HPI, HPI fragment, HPI-related polypeptide, a fragment of a HPI-related polypeptide, or a HPI fusion protein can be determined by methods known to those of skill in the art. These assays, whether cell-based or cell-free, can be used to screen a plurality (e.g., a library) of candidate agents.

In another embodiment, agents that modulate (i.e., upregulate or downregulate) the expression of a HPI, or a HPI-related polypeptide are identified by contacting cells (e.g., cells of prokaryotic origin or eukaryotic origin) expressing the HPI, or HPI-related polypeptide with a candidate agent or a control agent (e.g., phosphate buffered saline (PBS)) and determining the expression of the HPI, HPI-related polypeptide, or HPI fusion protein, mRNA encoding the HPI, or mRNA encoding the HPI-related polypeptide. The level of expression of a selected HPI, HPI-related polypeptide, mRNA encoding the HPI, or mRNA encoding the HPI-related polypeptide in the presence of the candidate agent is compared to the level of expression of the HPI, HPI-related polypeptide, mRNA encoding the HPI, or mRNA encoding the HPI-related polypeptide in the absence of the candidate agent (e.g., in the presence of a control agent). The candidate agent can then be identified as a modulator of the expression of the HPI, or a HPI-related polypeptide based on this comparison. For example, when expression of the HPI or mRNA is significantly greater in the presence of the candidate agent than in its absence, the candidate agent is identified as a stimulator of expression of the HPI or mRNA. Alternatively, when expression of the HPI or mRNA is significantly less in the presence of the candidate agent than in its absence, the candidate agent is identified as an inhibitor of the expression of the HPI or mRNA. The level of expression of a HPI or the mRNA that encodes it can be determined by methods known to those of skill in the art based on the present description. For example, mRNA expression can be assessed by Northern blot analysis or RT-PCR, and protein levels can be assessed by western blot analysis.

In another embodiment, agents that modulate the activity of a HPI, or a HPI-related polypeptide are identified by contacting a preparation containing the HPI or HPI-related polypeptide, or cells (e.g., prokaryotic or eukaryotic cells) expressing the HPI or HPI-related polypeptide with a candidate agent or a control agent and determining the ability of the candidate agent to modulate (e.g., stimulate or inhibit) the activity of the HPI or HPI-related polypeptide. The activity of a HPI or a HPI-related polypeptide can be assessed by detecting induction of a cellular signal transduction pathway of the HPI or HPI-related polypeptide (e.g., intracellular Ca²⁺, diacylglycerol, IP3, etc.), detecting catalytic or enzymatic activity of the target on a suitable substrate, detecting the induction of a reporter gene ( e.g., a regulatory element that is responsive to a HPI or a HPI-related polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities (see, e.g., U.S. Pat. No. 5,401,639, which is incorporated in its entirety herein by reference). The candidate agent can then be identified as a modulator of the activity of a HPI or HPI-related polypeptide by comparing the effects of the candidate agent to the control agent. Suitable control agents include phosphate buffered saline (PBS) and normal saline (NS).

In another embodiment, agents that modulate (i.e., upregulate or downregulate) the expression, activity or both the expression and activity of a HPI or HPI-related polypeptide are identified in an animal model. Examples of suitable animals include, but are not limited to, mice, rats, rabbits, monkeys, guinea pigs, dogs and cats. Preferably, the animal used represent a model of hepatoma. In accordance with this embodiment, the candidate agent or a control agent is administered (e.g., orally, rectally or parenterally such as intraperitoneally or intravenously) to a suitable animal and the effect on the expression, activity or both expression and activity of the HPI or HPI-related polypeptide is determined. Changes in the expression of a HPI or HPI-related polypeptide can be assessed by any suitable method described above, based on the present description.

In yet another embodiment, a HPI or HPI-related polypeptide is used as a “bait protein” in a two-hybrid assay or three hybrid assay to identify other proteins that bind to or interact with a HPI or HPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the HPIs of the invention as, for example, upstream or downstream elements of a signaling pathway involving the HPIs of the invention.

This invention further provides novel agents identified by the above-described screening assays and uses thereof for treatments as described herein.

### 5.14 Therapeutic Uses of HPIs

The invention provides for treatment or prevention of various diseases and disorders by administration of a therapeutic agent. Such agents include but are not limited to: HPIs, HPI analogs, HPI-related polypeptides and derivatives (including fragments) thereof; antibodies to the foregoing; nucleic acids encoding HPIs, HPI analogs, HPI-related polypeptides and fragments thereof; antisense nucleic acids to a gene encoding a HPI or HPI-related polypeptide; and modulator (e.g., agonists and antagonists) of a gene encoding a HPI or HPI-related polypeptide. An important feature of the present invention is the identification of genes encoding HPIs involved in hepatoma. Hepatoma can be treated (e.g. to ameliorate symptoms or to retard onset or progression) or prevented by administration of a therapeutic agent that promotes function or expression of one or more HPIs that are decreased in the tissue or blood/serum of subjects having hepatoma, or by administration of a therapeutic agent that reduces function or expression of one or more HPIs that are increased in the tissue or blood/serum of subjects having hepatoma.

In one embodiment, one or more antibodies each specifically binding to a HPI are administered alone or in combination with one or more additional therapeutic compounds or treatments.

Preferably, a biological product such as an antibody is allogeneic to the subject to which it is administered. In a preferred embodiment, a human HPI or a human HPI-related polypeptide, a nucleotide sequence encoding a human HPI or a human HPI-related polypeptide, or an antibody to a human HPI or a human HPI-related polypeptide, is administered to a human subject for therapy (e.g. to ameliorate symptoms or to retard onset or progression) or prophylaxis.

### 5.14.1 Treatment and Prevention of Hepatoma

Hepatoma can be treated or prevented by administration to a subject suspected of having or known to have hepatoma or to be at risk of developing hepatoma of an agent that modulates (i.e., increases or decreases) the level or activity (i.e., function) of one or more HPIs or the level of one or more HFs that are differentially present in the tissue or blood/serum of subjects having hepatoma compared with tissue or blood/serum of subjects free from hepatoma. In one embodiment, hepatoma is treated by administering to a subject suspected of having or known to have hepatoma or to be at risk of developing hepatoma an agent that upregulates (i.e., increases) the level or activity (i.e., function) of one or more HPIs or the level of one or more HFs that are decreased in the tissue or blood/serum of subjects having hepatoma. In another embodiment, an agent is administered that downregulates the level or activity (i.e., function) of one or more HPIs or the level of one or more HFs that are increased in the tissue or blood/serum of subjects having hepatoma. Examples of such an agent include but are not limited to: HPIs, HPI fragments and HPI-related polypeptides; nucleic acids encoding a HPI, a HPI fragment and a HPI-related polypeptide (e.g., for use in gene therapy); and, for those HPIs or HPI-related polypeptides with enzymatic activity, compounds or molecules known to modulate that enzymatic activity. Other agents that can be used, e.g., HPI agonists, can be identified using in vitro assays, as defined or described above or earlier.

Hepatoma is also treated or prevented by administration to a subject suspected of having or known to have hepatoma or to be at risk of developing hepatoma of an agent that downregulates the level or activity of one or more HPIs or the level of one or more HFs that are increased in the tissue or blood/serum of subjects having hepatoma. In another embodiment, an agent is administered that upregulates the level or activity of one or more HPIs or the level of one or more HFs that are decreased in the tissue or blood/serum of subjects having hepatoma. Examples of such an agent include, but are not limited to, HPI antisense oligonucleotides, ribozymes, antibodies directed against HPIs, and agents that inhibit the enzymatic activity of a HPI. Other useful agents e.g., HPI antagonists and small molecule HPI antagonists, can be identified using in vitro assays.

In a preferred embodiment, therapy or prophylaxis is tailored to the needs of an individual subject. Thus, in specific embodiments, compounds that promote the level or function of one or more HPIs, or the level of one or more HFs, are therapeutically or prophylactically administered to a subject suspected of having or known to have hepatoma, in whom the levels or functions of said one or more HPIs, or levels of said one or more HFs, are absent or are decreased relative to a control or normal reference range. In further embodiments, agents that promote the level or function of one or more HPIs, or the level of one or more HFs, are therapeutically or prophylactically administered to a subject suspected of having or known to have hepatoma in whom the levels or functions of said one or more HPIs, or levels of said one or more HFs, are increased relative to a control or to a reference range. In further embodiments, agents that decrease the level or function of one or more HPIs, or the level of one or more HFs, are therapeutically or prophylactically administered to a subject suspected of having or known to have hepatoma in whom the levels or functions of said one or more HPIs, or levels of said one or more HFs, are increased relative to a control or to a reference range. In further embodiments, agents that decrease the level or function of one or more HPIs, or the level of one or more HFs, are therapeutically or prophylactically administered to a subject suspected of having or known to have hepatoma in whom the levels or functions of said one or more HPIs, or levels of said one or more HFs, are decreased relative to a control or to a reference range. The change in HPI function or level, or HF level, due to the administration of such agents can be readily detected, e.g., by obtaining a sample (e.g., a sample of tissue or blood/serum, blood or urine or a tissue sample such as biopsy tissue) and assaying in vitro the levels of said HFs or the levels or activities of said HPIs, or the levels of mRNAs encoding said HPIs, or any combination of the foregoing. Such assays can be performed before and after the administration of the agent as described herein.

The agents of the invention include but are not limited to any agent, e.g., a small organic molecule, protein, peptide, antibody, nucleic acid, etc. that restores the hepatoma HPI or HF profile towards normal.

### 5.14.2 Gene Therapy

In another embodiment, nucleic acids comprising a sequence encoding a HPI, a HPI fragment, HPI-related polypeptide or fragment of a HPI-related polypeptide, are administered to promote HPI function by way of gene therapy. Gene therapy refers to the administration of an expressed or expressible nucleic acid to a subject. In this embodiment, the nucleic acid produces its encoded polypeptide and the polypeptide mediates a therapeutic effect by promoting HPI function.

Any suitable methods for gene therapy available in the art can be used according to the present invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, N.Y.

In a particular aspect, the agent comprises a nucleic acid encoding a HPI or fragment or chimeric protein thereof, said nucleic acid being part of an expression vector that expresses a HPI or fragment or chimeric protein thereof in a suitable host. In particular, such a nucleic acid has a promoter operably linked to the HPI coding region, said promoter being inducible or constitutive (and, optionally, tissue-specific). In another particular embodiment, a nucleic acid molecule is used in which the HPI coding sequences and any other desired sequences are flanked by regions that promote homologous recombination at a desired site in the genome, thus providing for intrachromosomal expression of the HPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a further embodiment, a viral vector that contains a nucleic acid encoding a HPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the HPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a HPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid to be introduced for purposes of gene therapy may comprise an inducible promoter operably linked to the coding region, such that expression of the nucleic acid is controllable by controlling the presence or absence of the appropriate inducer of transcription.

Direct injection of a DNA coding for a HPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a HPI and (b) a promoter are injected into a subject to elicit an immune response to the HPI.

### 5.14.3 Inhibition of HPIs to Treat Hepatoma

In one embodiment of the invention, hepatoma is treated or prevented by administration of an agent that antagonizes (inhibits) the level(s) and/or function(s) of one or more HPIs which are elevated in the tissue or blood/serum of subjects having hepatoma as compared with tissue or blood/serum of subjects free from hepatoma. Agents useful for this purpose include but are not limited to anti-HPI antibodies (and fragments and derivatives containing the binding region thereof), HPI antisense or ribozyme nucleic acids, and nucleic acids encoding dysfunctional HPIs that are used to “knockout” endogenous HPI function by homologous recombination (see, e.g., Capecchi, 1989, Science 244:1288-1292). Other agents that inhibit HPI function can be identified by use of known in vitro assays, e.g., assays for the ability of a candidate agent to inhibit binding of a HPI to another protein or a binding partner, or to inhibit a known HPI function. Preferably such inhibition is assayed in vitro or in cell culture, but genetic assays may also be employed. The Preferred Technology can also be used to detect levels of the HPI before and after the administration of the agent. Preferably, suitable in vitro or in vivo assays are utilized to determine the effect of a specific agent and whether its administration is indicated for treatment of the affected tissue, as described in more detail below.

In a particular embodiment, an agent that inhibits a HPI function is administered therapeutically or prophylactically to a subject in whom an increased tissue or blood/serum level or functional activity of the HPI (e.g., greater than the normal level or desired level) is detected as compared with tissue or blood/serum of subjects free from hepatoma or a predetermined reference range. Methods standard in the art can be employed to measure the increase in a HPI level or function, as outlined above. Preferred HPI inhibitor compositions include small molecules, i.e., molecules of 1000 daltons or less. Such small molecules can be identified by the screening methods described herein.

### 5.14.4 Antisense Regulation of HPIs

In a further embodiment, HPI expression is inhibited by use of HPI antisense nucleic acids. The present invention provides the therapeutic or prophylactic use of nucleic acids comprising at least six nucleotides that are antisense to a gene or cDNA encoding a HPI or a portion thereof As used herein, a HPI “antisense” nucleic acid refers to a nucleic acid capable of hybridizing by virtue of some sequence complementarity to a portion of an RNA (preferably mRNA) encoding a HPI. The antisense nucleic acid may be complementary to a coding and/or noncoding region of an mRNA encoding a HPI. Such antisense nucleic acids have utility as compounds that inhibit HPI expression, and can be used in the treatment or prevention of hepatoma.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of a HPI antisense nucleic acid, and a pharmaceutically-acceptable carrier, vehicle or diluent.

In another embodiment, the invention provides methods for inhibiting the expression of a HPI nucleic acid sequence in a prokaryotic or eukaryotic cell comprising providing the cell with an effective amount of a composition comprising a HPI antisense nucleic acid of the invention.

HPI antisense nucleic acids and their uses are described in detail below.

### 5.14.5 HPI Antisense Nucleic Acids

The HPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a particular aspect of the invention, a HPI antisense oligonucleotide is provided, preferably of single-stranded DNA. The oligonucleotide may be modified at any position on its structure with substituents generally known in the art.

The HPI antisense oligonucleotide may comprise any suitable of the following modified base moieties, e.g. 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxynracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In another embodiment, the HPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the HPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the HPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a HPI, preferably a human gene encoding a HPI. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.) with the RNA, forming a stable duplex; in the case of double-stranded HPI antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding a HPI it may contain and still form a stable duplex (or triplex, as the case may be). One skilled in the art can ascertain a tolerable degree of mismatch by use of standard procedures to determine the melting point of the hybridized complex.

### 5.14.6 Therapeutic Use of HPI Antisense Nucleic Acids

The HPI antisense nucleic acids can be used to treat or prevent hepatoma when the target HPI is overexpressed in the tissue or blood/serum of subjects suspected of having or suffering from hepatoma. In a preferred embodiment, a single-stranded DNA antisense HPI oligonucleotide is used.

Cell types which express or over-express RNA encoding a HPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with a HPI-specific nucleic acid (e.g., by Northern hybridization, dot blot hybridization, in situ hybridization), observing the ability of RNA from the cell type to be translated in vitro into a HPI, immunoassay, etc. In a preferred aspect, primary tissue from a subject can be assayed for HPI expression prior to treatment, e.g., by immunocytochemistry or in situ hybridization.

Pharmaceutical compositions of the invention, comprising an effective amount of a HPI antisense nucleic acid in a pharmaceutically acceptable carrier, vehicle or diluent can be administered to a subject having hepatoma.

The amount of HPI antisense nucleic acid which will be effective in the treatment of hepatoma can be determined by standard clinical techniques.

In a specific embodiment, pharmaceutical compositions comprising one or more HPI antisense nucleic acids are administered via liposomes, microparticles, or microcapsules. In various embodiments of the invention, such compositions may be used to achieve sustained release of the HPI antisense nucleic acids.

### 5.14.7 Inhibitory Ribozyme and Triple Helix Approaches

In another embodiment, symptoms of hepatoma may be ameliorated by decreasing the level of a HPI or HPI activity by using gene sequences encoding the HPI in conjunction with well-known gene “knock-out,” ribozyme or triple helix methods to decrease gene expression of a HPI. In this approach ribozyme or triple helix molecules are used to modulate the activity, expression or synthesis of the gene encoding the HPI, and thus to ameliorate the symptoms of hepatoma. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding a HPI can be used to prevent translation of target gene mRNA and, therefore, expression of the gene product. (See, e.g., PCT International Publication WO90/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding a HPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the HPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the HPI.

As in the antisense approach, the ribozymes can be composed of modified oligonucleotides (e.g., for improved stability, targeting, etc.) and should be delivered to cells that express the HPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the HPI and inhibit translation. Because ribozymes, unlike antisense molecules, are catalytic, a lower intracellular concentration is required for efficacy.

Endogenous HPI expression can also be reduced by inactivating or “knocking out” the gene encoding the HPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional HPI (or a completely unrelated DNA sequence) flanked by DNA homologous to the endogenous gene (either the coding regions or regulatory regions of the gene encoding the HPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However, this approach can be adapted for use in humans provided the recombinant DNA constructs are directly administered or targeted to the required site in vivo using appropriate viral vectors.

Alternatively, the endogenous expression of a gene encoding a HPI can be reduced by targeting deoxyribonucleotide sequences complementary to the regulatory region of the gene (i.e., the gene promoter and/or enhancers) to form triple helical structures that prevent transcription of the gene encoding the HPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription in the present invention should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In one embodiment, wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of a HPI that the situation may arise wherein the concentration of HPI present may be lower than is necessary for a normal phenotype. In such cases, to ensure that substantially normal levels of activity of a gene encoding a HPI are maintained, gene therapy may be used to introduce into cells nucleic acid molecules that encode and express the HPI that exhibit normal gene activity and that do not contain sequences susceptible to whatever antisense, ribozyme, or triple helix treatments are being utilized. Alternatively, in instances whereby the gene encodes an extracellular protein, normal HPI can be co-administered in order to maintain the requisite level of HPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### 5.15 Assays for Therapeutic or Prophylactic Compounds

The present invention also provides assays for use in discovery of pharmaceutical products in order to identify or verify the efficacy of agents for treatment or prevention of hepatoma. Agents can be assayed for their ability to restore HF or HPI levels in a subject having hepatoma towards levels found in subjects free from hepatoma or to produce similar changes in experimental animal models of hepatoma. Agents able to restore HF or HPI levels in a subject having hepatoma towards levels found in subjects free from hepatoma or to produce similar changes in experimental animal models of hepatoma can be used as lead agents for further drug discovery, or used therapeutically. HF and HPI expression can be assayed by the Preferred Technology, immunoassays, gel electrophoresis followed by visualization, detection of HPI activity, or any other method taught herein or known to those skilled in the art. Such assays can be used to screen candidate agents, in clinical monitoring or in drug development, where abundance of an HF or HPI can serve as a surrogate marker for clinical disease.

In various embodiments, in vitro assays can be carried out with cells representative of cell types involved in a subject's disorder, to determine if an agent has a desired effect upon such cell types.

Agents for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. It is also apparent to the skilled artisan that, based upon the present disclosure, transgenic animals can be produced with “knock-out” mutations of the gene or genes encoding one or more HPIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, candidate agents that modulate the expression of a HPI are identified in non-human animals (e.g., mice, rats, monkeys, rabbits, and guinea pigs), preferably non-human animal models for hepatoma, expressing the HPI. In accordance with this embodiment, a candidate agent or a control agent is administered to the animals, and the effect of the candidate agent on expression of one or more HPIs is determined. A candidate agent that alters the expression of a HPI (or a plurality of HPIs) can be identified by comparing the level of the selected HPI or HPIs (or mRNA(s) encoding the same) in an animal or group of animals treated with a candidate agent with the level of the HPI(s) or mRNA(s) in an animal or group of animals treated with a control agent. Techniques known to those of skill in the art can be used to determine the mRNA and protein levels, for example, in situ hybridization. The animals may or may not be sacrificed to assay the effects of a candidate agent.

In another embodiment, candidate agents that modulate the activity of a HPI or a biologically active portion thereof are identified in non-human animals (e.g., mice, rats, monkeys, rabbits, and guinea pigs), preferably non-human animal models for hepatoma, expressing the HPI. In accordance with this embodiment, a candidate agent or a control agent is administered to the animals, and the effect of a candidate agent on the activity of a HPI is determined. A candidate agent that alters the activity of a HPI (or a plurality of HPIs) can be identified by assaying animals treated with a control agent and animals treated with the candidate agent. The activity of the HPI can be assessed by detecting induction of a cellular second messenger of the HPI (e.g., intracellular Ca²⁺, diacylglycerol, IP3, etc.), detecting catalytic or enzymatic activity of the HPI or binding partner thereof, detecting the induction of a reporter gene (e.g., a regulatory element that is responsive to a HPI of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a HPI (see, e.g., U.S. Pat. No. 5,401,639, which is incorporated herein in its entirety by reference).

In yet another embodiment, candidate agents that modulate the level or expression of a HPI (or plurality of HPIs) are identified in human subjects having hepatoma, most preferably those having severe or metastatic hepatoma. In accordance with this embodiment, a candidate agent or a control agent is administered to the human subject, and the effect of a candidate agent on HPI expression is determined by analyzing the expression of the HPI or the mRNA encoding the same in a biological sample (e.g., tissue or blood/serum, serum, plasma, or urine]). A candidate agent that alters the expression of a HPI can be identified by comparing the level of the HPI or mRNA encoding the same in a subject or group of subjects treated with a control agent to that in a subject or group of subjects treated with a candidate agent. Alternatively, alterations in the expression of a HPI can be identified by comparing the level of the HPI or mRNA encoding the same in a subject or group of subjects before and after the administration of a candidate agent. Any suitable techniques known to those of skill in the art can be used to obtain the biological sample and analyze the mRNA or protein expression. For example, the Preferred Technology described herein can be used to assess changes in the level of a HPI.

In another embodiment, candidate agents that modulate the activity of a HPI (or plurality of HPIs) are identified in human subjects having hepatoma, most preferably those with severe or metastatic hepatoma. In this embodiment, a candidate agent or a control agent is administered to the human subject, and the effect of a candidate agent on the activity of a HPI is determined. A candidate agent that alters the activity of a HPI can be identified by comparing biological samples from subjects treated with a control agent to samples from subjects treated with the candidate agent. Alternatively, alterations in the activity of a HPI can be identified by comparing the activity of a HPI in a subject or group of subjects before and after the administration of a candidate agent. The activity of the HPI can be assessed by detecting in a biological sample (e.g., tissue or blood/serum, serum, plasma, or urine) induction of a downstream effector e.g. a cellular signal transduction pathway of the HPI (e.g., intracellular Ca²⁺, diacylglycerol, IP3, etc.), catalytic or enzymatic activity of the HPI or a binding partner thereof, or a cellular response, for example, cellular differentiation, or cell proliferation. Techniques known to those of skill in the art can be used to detect changes in the induction of a second messenger of a HPI or changes in a cellular response. For example, RT-PCR can be used to detect changes in the induction of a cellular second messenger.

In a particular embodiment, an agent that changes the level or expression of a HPI towards levels detected in control subjects (e.g., humans free from hepatoma) is selected for further testing or therapeutic use. In another preferred embodiment, a candidate agent that changes the activity of a HPI towards the activity found in control subjects (e.g., humans free from hepatoma) is selected for further testing or therapeutic use.

In another embodiment, candidate agents that reduce the severity of one or more symptoms associated with hepatoma are identified in human subjects having hepatoma, most preferably subjects with severe or metastatic hepatoma. In accordance with this embodiment, a candidate agent or a control agent is administered to the subjects, and the effect of a candidate agent on one or more symptoms of hepatoma is determined. A candidate agent that reduces one or more symptoms can be identified by comparing the subjects treated with a control agent to the subjects treated with the candidate agent. Techniques known to physicians familiar with hepatoma can be used to determine whether a candidate agent reduces one or more symptoms associated with hepatoma. For example, a candidate agent that reduces tumour size in a subject having hepatoma will be beneficial for treating subjects having hepatoma.

In a preferred embodiment, an agent that reduces the severity of one or more symptoms associated with hepatoma in a human having hepatoma is selected for further testing or therapeutic use. cl 5.16 Therapeutic and Prophylactic Compositions and Their Use

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or othersvector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into tissue or blood/serum or at the site (or former site) of tumorigenesis.

In another embodiment, the agent can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the agent can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the liver, thus requiring only a fraction of the systemic dose (see, e.g., Goodson, in Medical Applications of Controlled Release, supra, vol. 2, pp. 115-138 (1984)).

Other suitable controlled release systems are discussed in the review by Langer (1990, Science 249:1527-1533).

In another embodiment where the agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of hepatoma can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### 6. Example: Proteins from Serum of Patients with and without Hepatoma

Using the following Reference Protocol, proteins in serum from 33 patients with hepatoma and 19 patients with cirrhosis of the liver were separated by isoelectric focusing followed by SDS-PAGE and analyzed. Each sample was run in duplicate.

### 6.1. Sample preparation

A protein assay was carried out on the serum sample as received (Pierce BCA Cat # 23225). A volume of serum corresponding to 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2×)

This mixture was vortexed, and centrifuged at 13000 rpm for 5 mins at 15° C., and the supernatant was analyzed by isoelectric focusing.

### 6.2. Isoelectric Focusing

Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For JEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs For all stages of the process, the current limit was set to 10 mA for 12 gels, and the wattage limit to 5W. The temperature was held at 20° C. throughout the run.

### 6.3. Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma I-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

### 6.4. Preparation of supported gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of y-methacryl-oxypropyltrimethoxysilane in ethanol (BindSilane™; Pharnacia Cat. # 17-1330-01). The front plate was treated with a 2% solution of dimethyldichlorosilane dissolved in octamethyl cyclo-octasilane (RepelSilane™, Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

### 6.5. SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 10° C. throughout the run.

### 6.6. Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion in a staining solution for 4 hours. A solution of fluorescent dye was prepared by diluting Sypro Red (Molecular Probes, Inc., Eugene, Ore.) according to the manufacturer's instructions; this diluted solution was filtered under vacuum though a 0.4 μm filter.

### 6.7. Imaging of the gel

A computer-readable output was produced by imaging the fluorescently stained gels with a Storm scanner (Molecular Dynamics, Sunnyvale, Calif.) according to the manufacturer's instructions, (see Storm User's Guide, 1995, Version 4.0, Part No. 149-355, incorporated herein by reference in its entirety) with modifications as described below. The gels were removed from the stain, rinsed with water briefly, and imaged on the Storm Scanner, in Red Fluorescence mode with a PMT setting of 1000V, and a resolution of 200 μm. Since the gel was rigidly bonded to a glass plate, the gel was held in contact with the scanner bed during imaging. To avoid interference patterns arising from non-uniform contact between the gel and the scanner bed, a film of water was introduced under the gel, taking care to avoid air pockets. Moreover, the gel was placed in a frame provided with two fluorescent buttons that were imaged together with the gel to provide reference points (designated M1 and M2) for determining the x,y coordinates of other features detected in the gel. A matched frame was provided on a robotic gel excisor in order to preserve accurate alignment of the gel. After imaging, the gels were sealed in polyethylene bags containing a small volume of staining solution, and then stored at 4° C.

### 6.8. Digital Analysis of the Data

The data were processed as described in U.S. Pat. No. 6,278,794, Sections 5.4 and 5.5 (incorporated herein by reference), as set forth more particularly below.

### 6.8.1. Computer Analysis Of The Detector Output

The output from the scanner was first processed using the MELANIE® II 2D PAGE analysis program (Release 2.2, 1997, BioRad Laboratories, Hercules, Calif., Cat. # 170-7566) to autodetect the registration points, M1 and M2; to autocrop the images (i.e., to eliminate signals originating from areas of the scanned image lying outside the boundaries of the gel, e.g., the reference frame); to filter out artifacts due to dust; to detect and quantify features; and to create image files in GJF format. Features were detected using the following parameters:

Smooths=2

Laplacian threshold 50

Partials threshold 1

Saturation=100

Peakedness=0

Minimum Perimeter=10

### 6.9. Assignment of PI and MW Values

Images were evaluated to reject images which had gross abnormalities, or were of too low a loading or overall image intensity, or were of too poor a resolution, or where duplicates were too dissimilar. If one image of a duplicate was rejected then the other image belonging to the duplicate was also rejected regardless of image quality. Samples that were rejected were scheduled for repeat analysis.

Landmark identification was used to determine the pI and MW values of features detected in the images. This process involves the identification of certain proteins which are expected to be found in any given biological sample. As these common proteins exhibit an identical isoelectric point and molecular weight from sample to sample, they can be used as standards; this process also corrects for any possible gel variation or distortion.

From the dataset of normal serum gels, a gel was arbitrarily chosen as the Primary Master Gel. Landmark features were then identified by comparing the features detected in this Primary Master Gel with features previously identified on 2D electrophoresis of normal human serum. (see Bjellqvist et al., 1993, Electrophoresis 14: 1357-1365; incorporated herein by reference in its entirety).

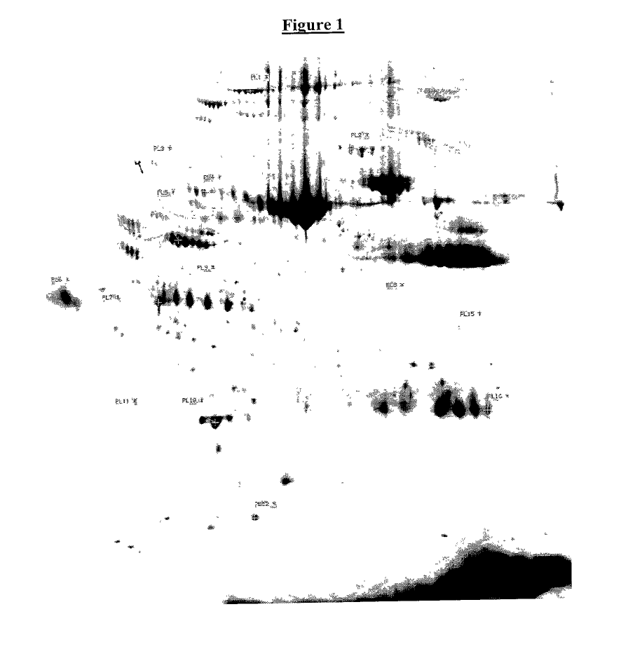

Fourteen landmark features, designated PL1 to PL 12 and PL 15 to PL16, were identified in the Primary Master Gel. These landmark features are identified in FIG. 1 and were assigned the pI and/or MW values indicated in Table VII.

TABLE VII
Landmark Features used in this study

| | | MW |
|---|---|---|
| Name | pI | (kd) |
| PL1 | None | 186,073 |
| PL2 | 6.20 | 100,000 |
| PL3 | 4.73 | 93,708 |
| PL4 | 5.13 | 73,465 |
| PL5 | 4.97 | 52,739 |
| PL6 | 4.10 | None |
| PL7 | 4.80 | 40,997 |
| PL8 | 6.47 | 47,195 |
| PL9 | 5.29 | 43,541 |
| PL10 | 5.22 | 23,000 |
| PL11 | 4.47 | 25,183 |
| PL12 | 5.52 | 13,800 |
| PL15 | 7.80 | 36,962 |
| PL16 | 8.58 | None |

As many of these landmarks as possible were identified in each gel image in the dataset.

All features in the Master geI were then assigned a pI value by linear interpolation/extrapolation (using the MELANIE II software) to the pI of the two nearest landmarks that had been assigned a pI value, and were assigned a MW value by linear interpolation/extrapolation (using the MELANIE II software) to the MW of the two nearest landmarks that had been assigned a MW value. Each feature was also labelled with a unique number known as its Molecular Cluster Index (or MCI).

Secondary Master gels were chosen for both the cirrhosis gels and the HCC gels. Features in these gels were paired with common features in the Master gel, using the algorithm supplied with the MELANIE II software, as described at Section A, pp. 8-10 of the MELANIE II 2D PAGE (Release 2.2) User Manual (The Melanie Group, Geneva, Switzerland). Features that have been paired are linked to the corresponding MCI, and hence to an associated pI and MW value. Unpaired features present in these secondary master gels were assigned pI and MW values by linear interpolation/extrapolation (using the MELANIE II software) with respect to pI and MW of the landmarks. Additional unique entries were then created in the MCI for these features.

### 6.9.1. Construction of Profiles

All gels in the dataset were now matched to the Primary and Secondary Master Gels, and paired features were linked to the corresponding entries in the Molecular Cluster Index.

Duplicate gels were then aligned via the landmarks and a matching process performed so as to pair identical spots on the duplicate gels. This provided increased assurance that subsequently measured isoelectric points and molecular weights were accurate, as paired spots demonstrated the reproducibility of the separation and also filtered out artefacts.

A measurement of the intensity of each protein spot was taken and stored. Each protein spot was assigned an identification code and matched to a spot on the Master gel.

The end result of this aspect of the analysis was the generation, for each duplicate set of gels representing a single serum sample, of a digital profile which contained, for each identified spot: 1) a unique arbitrary identification code, 2) the x, y coordinates, 3) the isoelectric point, 4) the molecular weight, 5) the signal value, 6) the standard deviation for each of the preceding measurements, and 7) a pointer to the MCI of the spot on the master gel to which this spot was matched. By virtue of the Laboratory Information Management System (LIMS), this profile was traceable to the actual stored gel from which it was generated, so that proteins identified by computer analysis of gel profile databases could be retrieved. The LIMS also permitted the profile to be traced back to the original sample or patient.

### 6.9.2. Cross-Matching Between Samples

Once the profile was generated, analysis was directed toward the selection of interesting proteins. Each significant feature in a profile was assigned an index, the “Molecular Cluster Index” (MCI) that identifies the feature in all gels and that serves as a pointer to parameters (1) to (7) above of the feature. A molecular cluster table was generated from the master gel for each sample type (i.e. hepatoma serum and cirrhosis serum). Gels from all other samples of the same type were matched with the relevant primary and secondary master gels. The digital profiles for each sample were then annotated by adding, for each matched feature, the MCI assigned to that feature in the master profile.

### 6.9.3. Differential Analysis of the Profiles

Within each sample set (hepatoma serum or cirrhosis serum), the profiles were analyzed to identify and select those features present in at least 50% of the profiles. These selected features were then assembled into a hepatoma serum feature set and a cirrhosis serum feature set. Matching features of each feature set were then compared to identify those features showing at least a 2-fold difference in mean intensity between hepatoma serum and cirrhosis serum. Differentially present features were identified as Hepatoma-Diagnostic Features (HFs).

### 6.9.4. Statistical Analysis Of HFs

For each HF, a statistical analysis was performed using Student's t-test to compare the distribution of signal intensities for that feature in the 33 hepatoma serum profiles and in the 19 cirrhosis serum profiles. A p value was derived for each HF.

### 6.10. Recovery and analysis of selected proteins

Proteins in HFs were robotically excised and processed to generate tryptic peptides using the Preferred Technology as described in U.S. application Ser. No. 08/980,574, which is incorporated herein by reference in its entirety. Partial amino acid sequences of these peptides were determined by mass spectroscopy, using de novo sequencing.

### 6.11. Results

These initial experiments identified 17 features that were decreased and 44 features that were increased in hepatoma serum as compared with cirrhosis serum. Details of these HFs and a p value for each are provided in Tables I and II. Each HF was differentially present in hepatoma serum as compared with non-hepatoma serum (p<0.1). For some preferred HFs (HF-1, HF-3, HF-10, HF-15, HF-16, HF-17, HF-18, HF-19, HF-20, HF-21, HF-23, HF-24, HF-25, HF-26, HF-28, HF-29, HF-31, HF-32, HF-33, HF-34, HF-36, HF-37, HF-38, HF-39, HF-40, HF-42, HF-44, HF-46, HF-47, HF-48, HF-49, HF-51, HF-52, HF-53, HF-54, HF-55, HF-56, HF-59, HF-60, HF-63, HF-75, HF-84, HF-85, HF-89, HF-91, HF-97, HF-100, HF-103, HF-109, HF-110, HF-112, HF-113, HF-120, HF-121, HF-122, and HF-124), the difference was more significant (p≦0.01), and for certain highly preferred HFs (HF-1, HF-17, HF-18, HF-20, HF-21, HF-23, HF-24, HF-25, HF-28, HF-29, HF-31, HF-33, HF-34, HF-36, HF-39, HF-44, HF-51, HF-52, HF-54, HF-55, HF-59, HF-75, HF-84, HF-85, HF-89, HF-91, HF-97, HF-110, HF-113, HF-120, HF-121, HF-122, and HF-124), the difference was still more significant (p≦0.001).

Partial amino acid sequences were determined for the differentially present HPIs in these HFs. Details of these HPIs are provided in Tables III and IV. Computer searches of public databases identified at least 2 HPIs for which neither the partial amino acid sequence, nor any oligonucleotide encoding such a peptide sequence, was described in any public database examined. Table IV illustrates that several HPIs are isoforms of the same protein. For example, HPI-2, HPI-6, HPI-8, HPI-14, HPI-15, HPI-17 and HPI-18 are isoforms of complement factor 4; HPI-4, HPI-5 and HPI-12 are isoforms of complement factor 3; and HPI-19, HPI-20 and HPI-21 are isoforms of ceruloplasmin. These isoforms are thought to arise from differences in post-translational processing (e.g., glycosylation, phosphorylation, acylation or minimal proteolysis).

### 1.1. Preparation of immobilised Antibody Hi-Trap™ Triple Column Assemblies

Removal of albumin, haptoglobin, transferrin, alpha-1-antitrypsin alpha-2-macroglobulin and immunoglobin G (IgG) from human serum (‘serum depletion’) is achieved by performing an affinity chromatography purification step where the sample is passed through a series of ‘Hi-Trap’ columns which contain immobilised antibodies to selectively remove albumin, haptoglobin, transferrin, alpha-1-antitrypsin alpha-2-macroglobulin and protein G to selectively remove immunoglobin G. Two affinity columns in a tandem assembly are prepared by coupling antibodies to protein G-sepharose contained in Hi-Trap columns (Protein G-Sepharose Hi-Trap columns (1 ml) Pharmacia Cat. No. 17-0404-01). This is done by circulating the following solutions sequentially through the columns:

1. Dulbecco's Phosphate Buffered Saline (Gibco BRL Cat. No. 14190-094)

2. concentrated antibody solution

3. 200 mM sodium carbonate buffer, pH 8.35

4. Cross-Linking Solution (200 mM sodium carbonate buffer, pH 8.35, 20 mM dimethylpimelimidate)

5. 500 mM ethanolamine, 500 mM NaCl

A third (un-derivatised) protein G Hi-Trap column is then attached to the lower end of the tandem column assembly.

The chromatographic procedure is automated using an Akta Fast Protein Liquid Chromatography (FPLC) System such that a series of up to seven runs can be performed sequentially. The samples are passed through the series of 3 Hi-Trap columns in which the affinity chromatography media selectively bind the above proteins thereby removing them from the sample. Typically fractions (3 ml per tube) are collected of unbound material (‘Flowthru fractions’) that elutes through the column during column loading and washing stages and of bound proteins (‘Bound/Eluted fractions’) that are eluted by step elution with Immunopure Gentle Ag/Ab Elution Buffer (Pierce Cat. No. 21013). The eluate containing unbound material is collected in fractions which are pooled, desalted/concentrated by centrifugal ultrafiltration and stored to await further analysis by 2D PAGE.

The present invention is not to be limited in scope by the particular exemplified embodiments, which are intended as illustrations of single aspects of the invention. Indeed, various modifications of the invention in addition to those enumerated herein will be apparent to those skilled in the art from the foregoing description and accompanying drawings. Such modifications are intended to fall within the scope of the appended claims.

All publications cited herein are incorporated by reference in their entirety.

1. A method for screening, diagnosis or prognosis of hepatoma in a human subject for determining the stage or severity of hepatoma in a subject, for identifying a subject at risk of developing hepatoma, or for monitoring the effect of therapy administered to a subject having hepatoma, said method comprising:
(a) analyzing a test sample of body fluid from the subject by two-dimensional electrophoresis to generate a two-dimensional array of features, said array comprising at least one chosen Hepatoma-Diagnostic Feature (HF) selected from: HF-1, HF-2, HF-3, HF-4, HF-5, HF-6, HF-7, HF-8, HF-9, HF-10, HF-11, HF-12, HF-13, HF-14, HF-15, HF-16, HF-17, HF-18, HF-19, HF-20, HF-21, HF-22, HF-23, HF-24, HF-25, HF-26, HF-27, HF-28, HF-29, HF-30, HF-31, HF-32, HF-33, HF-34, HF-35, HF-36, HF-37, HF-38, HF-39, HF-40, HF-41, HF-42, HF-43, HF-44, HF-45, HF-46, HF-47, HF-48, HF-49, HF-50, HF-51, HF-52, HF-53, HF-54, HF-55, HF-56, HF-57, HF-58, HF-59, HF-60, HF-61, HF-62, HF-63, HF-64, HF-65, HF-66, HF-67, HF-68, HF-69, HF-70, HF-71, HF-72, HF-73, HF-74, HF-75, HF-76, HF-77, HF-78, HF-79, HF-80, HF-81, HF-82, HF-83, HF-84, HF-85, HF-86, HF-87, HF-88, HF-89, HF-90, HF-91, HF-92, HF-93, HF-94, HF-95, HF-96, HF-97, HF-98, HF-99, HF-100, HF-101, HF-102, HF-103, HF-104, HF-105, HF-106, HF-107, HF-108, HF-109, HF-110, HF-111, HF-112, HF-113, HF-114, HF-115, HF-116, HF-117, HF-118, HF-119, HF-120, HF-121, HF-122, HF-123, HF-124, HF-125, HF-126, HF-127, HF-128, HF-129, HF-130, HF-131, HF-132, HF-133, HF-134, HF-135, HF-136, HF-137, HF-138, HF-139, HF-140, HF-141; and
(b) comparing the abundance of each chosen feature in the test sample with the abundance of that chosen feature in body fluid from one or more persons free from hepatoma, or with a previously determined reference range for that feature in subjects free from hepatoma, or with the abundance at least one Expression Reference Feature (ERF) in the test sample.

2. The method according to claim 1, wherein step (a) comprises quantitatively detecting at least one of the following HFs: HF-1, HF-3, HF-10, HF-15, HF-16, HF-17, HF-18, HF-19, HF-20, HF-21, HF-23, HF-24, HF-25, HF-26, HF-28, HF-29, HF-31, HF-32, HF-33, HF-34, HF-36, HF-37, HF-38, HF-39, HF-40, HF-42, HF-44, HF-46, HF-47, HF-48, HF-49, HF-51, HF-52, HF-53, HF-54, HF-55, HF-56, HF-59, HF-60, HF-63, HF-75, HF-84, HF-85, HF-89, HF-91, HF-97, HF-100, HF-103, HF-109, HF-110, HF-112, HF-113, HF-120, HF-121, HF-122 and HF-124.

3. The method according to claim 2, wherein step (a) comprises quantitatively detecting at least one of the following HFs: HF-17, HF-18, HF-20, HF-23, HF-24, HF-25, HF-29, HF-33, HF-34, HF-39, HF-44, HF-55, HF-60, HF-75, HF-84, HF-85, HF-89, HF-91, HF-97, HF-110, HF-113, HF-120, HF-121, HF-122 and HF-124.

4. The method according to any one of claims 1 to 3, wherein the body fluid is serum or plasma.

5. The method according to claim 1, wherein step (b) comprises comparing the abundance of each chosen feature in the sample with the abundance of that chosen feature in serum or plasma from one or more persons free from hepatoma or with a previously determined reference range for that chosen feature in subjects free from hepatoma.

6. The method as claimed in claim 1, which is used in combination with an assay or assessment for Hepatitis B and/or Hepatitis C.

7. A method for screening, diagnosis or prognosis of hepatoma in a subject, for determining the stage or severity of hepatoma in a subject, for identifying a subject at risk of developing hepatoma, or for monitoring the effect of therapy administered to a subject having hepataoma, said method comprising quantitatively detecting, in a sample of serum or plasma from the subject, at least one of the following Hepatoma-Diagnostic Protein Isoforms (HPIs): HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27.

8. The method according to claim 7, wherein the step of quantitatively detecting comprises testing at least one aliquot of the sample, said step of testing comprising:
(a) contacting the aliquot with an antibody that is immunospecific for a preselected HPI; and
(b) detecting whether binding has occurred between the antibody and at least one species in the aliquot.

9. The method according to claim 8, wherein the step of quantitatively detecting comprises testing a plurality of aliquots with a plurality of antibodies for quantitative detection of a plurality of preselected HPIs.

10. An antibody capable of immunospecific binding to one of the following Hepatoma-Diagnostic Protein Isoforms (HPIs): HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27.

11. The antibody of claim 10, which is selected from monoclonal antibodies, chimeric antibodies, humanized antibodies and bispecific antibodies.

12. A preparation comprising at least one of the following isolated Hepatoma-Diagnostic Protein Isoforms (HPIs): HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27 or an antibody or a fragment or derivative of an antibody as claimed in claim 10, said fragment or derivative containing the binding domain of the antibody as claimed in claims 10, optionally with a pharmaceutically acceptable carrier.

13. A method of treating or preventing hepatoma comprising administering to a subject in need of such treatment or prevention a therapeutically effective amount of an antibody or a fragment or derivative of an antibody as claimed in claim 10, said fragment or derivative containing the binding domain of the antibody as claimed in claim 10 to 14.

14. The method of claim 13, wherein the antibody is used as a vaccine.

15. A method of treating or preventing hepatoma comprising administering to a subject in need of such treatment or prevention a therapeutically effective amount of a nucleic acid encoding one of the following Hepatoma-Diagnostic Protein Isoforms (HPIs): HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18,-HPI-19, HPI-20, HPI-21, HPI-22, HPI-23, HPI-24, HPI-25, HPI-26 and HPI-27.

16. A method of treating or preventing hepatoma comprising administering to a subject in need of such treatment or prevention a therapeutically effective amount of a nucleic acid that inhibits the expression or function of one or more of the following Hepatoma-Diagnostic Protein Isoforms (HPIs): HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27.

17. The method of claim 16, wherein the nucleic acid is a HPI antisense nucleic acid or ribozyme.

18. A method of screening for agents that interact with a Hepatoma-Diagnostic Protein Isoform (HPI), selected from HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27, or a HPI fragment, or HPI-related polypeptide said method comprising:
(a) contacting a HPI, a HPI fragment, or a HPI-related polypeptide with a candidate agent; and
(b) determining whether or not the candidate agent interacts with the HPI, the HPI fragment, or the HPI-related polypeptide.

19. A method of screening for agents that modulate the expression or activity of a Hepatoma-Diagnostic Protein Isoform (HPI), selected from: HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27, an HPI fragment, or an HPI-related polypeptide comprising:
(a) contacting a first population of cells expressing a HPI, a HPI fragment, or a HPI-related polypeptide with a candidate agent;
(b) contacting a second population of cells expressing said HPI, said HPI fragment, or said HPI-related polypeptide with a control agent; and
(c) comparing the level of said HPI, said HPI fragment, or said HPI-related polypeptide or mRNA encoding said HPI, said HPI fragment, or said HPI-related polypeptide in the first and second populations of cells, or comparing the level of induction of a cellular second messenger in the first and second populations of cells.

20. A method of screening for or identifying agents that modulate the expression or activity of a Hepatoma-Diagnostic Protein Isoform (HPI), selected from: HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27, an HPI fragment, or a HPI-related polypeptide comprising:
(a) administering a candidate agent to a first mammal or group of mammals;
(b) administering a control agent to a second mammal or group of mammals; and
(c) comparing the level of expression of the HPI, the HPI fragment, or the HPI-related polypeptide or of mRNA encoding the HPI, the HPI fragment, or the HPI-related polypeptide in the first and second groups, or comparing the level of induction of a cellular second messenger in the first and second groups; and
(d) optionally comparing the levels of expression of the HPI or the HPI-related polypeptide or of mRNA encoding the HPI or the HPI-related polypeptide in the first and second groups to the level of the HPI or the HPI-related polypeptide or of mRNA encoding the HPI or the HPI-related polypeptide in normal control mammals, or comparing the level of induction of a cellular second messenger in the first and second groups, to the level of induction of a cellular second messenger in normal control mammals.

21. The method of claim 20, wherein the mammals are animal models for hepatoma or human subjects having hepatoma.

22. A method of screening for or identifying agents that modulate the activity of a Hepatoma-Diagnostic Protein Isoform (HPI), selected from: HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27, an HPI fragment, or a HPI-related polypeptide, comprising
(a) in a first aliquot, contacting a candidate agent with the HPI, the HPI fragment, or the HPI-related polypeptide, and
(b) comparing the activity of the HPI, the HPI fragment, or the HPI-related polypeptide in the first aliquot after addition of the candidate agent with the activity of the HPI, the HPI fragment, or the HPI-related polypeptide in a control aliquot, or with a previously determined reference range.

23. The method according to claim 22, wherein the HPI, the HPI fragment, or the HPI-related polypeptide is recombinant protein.

24. The method according to claim 22, wherein the HPI, the HPI fragment, or the HPI-related polypeptide is immobilized on a solid phase.

25. An isolated nucleic acid molecule that hybridizes to a nucleotide sequence encoding HPI-13 or HPI-21 or their complements.

26. An isolated nucleic acid molecule that hybridizes to a nucleotide sequence encoding at least 10 consecutive amino acids of HPI-13 or HPI-21 or their complements.

27. A vector comprising the nucleic acid molecule of claim 25.

28. A host cell comprising the vector of claim 27.

29. A host cell genetically engineered to express the nucleic acid molecule of claim 25.

30. A method for screening, diagnosis or prognosis of hepatoma in a subject or for monitoring the effect of an anti-hepatoma drug or therapy administered to a subject, comprising:
(a) contacting at least one oligonucleotide probe comprising 10 or more consecutive nucleotides complementary to a nucleotide sequence encoding a HPI chosen from HPI-1, HPI-2, HPI-3, HPI-4, HPI-5, HPI-6, HPI-8, HPI-9, HPI-10, HPI-11, HPI-12, HPI-13, HPI-14, HPI-15, HPI-16, HPI-17, HPI-18, HPI-19, HPI-20, HPI-21, HPI-22, HPI-23, HPI-24, HPI-25, HPI-26 and HPI-27, with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

31. The method of claim 30, wherein step (a) includes the step of hybridizing the nucleotide sequence to a DNA array, wherein one or more members of the array are the probes complementary to a plurality of nucleotide sequences encoding distinct HPIs.
